(19) United States
(12) Patent Application Publication
(10) Pub. No.: US 2022/0127232 A1
(43) Pub. Date: Apr. 28, 2022

(54) HISTONE ACETYLTRANSFERASE MODULATORS AND COMPOSITIONS AND USES THEREOF

(71) Applicants: The Trustees of Columbia University in the City of New York, New York, NY (US); APPIA PHARMACEUTICALS LLC, New York, NY (US)

(72) Inventors: Ottavio ARANCIO, New York, NY (US); Elisa ZUCCARELLO, New York, NY (US); Elisa CALCAGNO, New York, NY (US); Donald W. LANDRY, New York, NY (US); Shixian DENG, White Plains, NY (US); Jole FIORITO, Floral Park, NY (US); Luuk Elard DE VRIES, Amsterdam (NL); Christopher John YARNOLD, Abingdon, Oxfordshire (GB); Richard Scott JONES, Abingdon, Oxfordshire (GB); Julian Hugh ROWLEY, Abingdon, Oxfordshire (GB)

(21) Appl. No.: 17/429,049

(22) PCT Filed: Feb. 7, 2020
(86) PCT No.: PCT/US20/17236

§ 371 Date: Aug. 6, 2021

Related U.S. Application Data
(60) Provisional application No. 62/803,195, filed on Feb. 8, 2019.

(51) Int. Cl. C07D 213/82; C07C 235/44; C07D 213/75; A61P 25/28

(52) U.S. Cl. CPC C07D 213/82; A61P 25/28; C07D 213/75; C07C 235/44

(57) ABSTRACT

Compounds and compositions comprising compounds that modulate histone acyl transferase (HAT). The invention further provides methods for treating neurodegenerative disorders, conditions associated with accumulated amyloid-beta peptide deposits, Tau protein levels, and/or accumulations of alpha-synuclein as well as cancer by administering a compound that modulates HAT to a subject.

### CROSS REFERENCE TO RELATED APPLICATIONS

This application claims the benefit of U.S. Provisional Patent Application No. 62/803,195, filed on Feb. 8, 2019, entitled “Histone Acetyltransferase (HAT) Regulators and Uses Thereof,” which is incorporated herein by reference.

### BACKGROUND

Modulation of the acetylation state of histones, transcription factors, and other regulatory proteins is known to influence their activity within neuronal, cancer and inflammatory cells. The acetylation state of a protein is controlled by the activity of two main groups of enzymes, histone deacetylases (HDAC) and histone acetyl transferases (HAT). The HDAC removes acetyl-groups while the HATs transfer acetyl-groups to the protein of interest.

Cognitive neurodegenerative disorders are characterized by synaptic dysfunction, cognitive abnormalities, and/or the presence of inclusion bodies throughout the CNS containing, for example, but not limited to native beta-amyloid fragments, native and phosphorylated Tau, native and phosphorylated alpha-synuclein, lipofuscin, cleaved TARDBP (TDB-43), oligomers of amyloid-beta (Aβ), tau and α-synuclein, in various percentages and in relation to the specific disease.

Alzheimer's disease (AD) is an irreversible neurodegenerative disease characterized by memory loss, synaptic dysfunction and accumulation of amyloid β-peptides (Aβ). The pathogenesis of AD is believed to be caused by high levels and aggregation of amyloid-β (Aβ) in the brain. AP has been found to impair memory by reducing acetylation of specific histone lysines important for memory formation. Histones are proteins that closely associate with DNA molecules and play an important role in gene transcription.

Currently available therapies for AD are palliative and do not cure the disease. Cholinesterase inhibitors such as Razadyne® (galantamine), Exelon® (rivastigmine), Aricept® (donepezil), and Cognex® (tacrine) have been prescribed for early stages of Alzheimer's disease, and may temporarily delay or prevent progression of symptoms related to AD. However, as AD progresses, the brain loses less acetylcholine, thereby rendering cholinesterase inhibitors unproductive as treatment for AD. Namenda® (memantine), an N-methyl D-aspartate (NMDA) antagonist, is also prescribed to treat moderate to severe Alzheimer's disease; however only temporary benefits are realized.

Classically, modulation of acetylation status is known to influence the condensation of chromatin. In cancer, histones are deacetylated maintaining a condensed chromatin structure, and a transcriptionally silenced state. This transcriptional inactivation is mediated by HDACs which remove acetyl groups from histone tails, maintain a condensed chromatic structure. Inhibitors of HDACs help maintain transcriptionally active chromatin, theoretically allowing for expression of tumor suppressor genes. One observation that has evolved is that histones are not the only targets of acetylation. It is now accepted that post-translational acetylation of intracellular proteins such as tumor suppressors (p53) and oncogenes (Bcl6) plays a critical role in influencing their activity. It has been established that there is a network of proteins and enzymes that can be modified by acetylation, now collectively referred to as the acetylome.

Histone Acetyltransferases (HATs) are involved in histone acetylation (leading to gene activation), chromosome decondensation, DNA repair and non-histone substrate modification. The post-translational acetylation status of chromatin is governed by the competing activities of two classes of enzymes, HATs and HDACs. The potential of inhibiting HDACs to counteract neurodegenerative disorders has been widely explored (Curr Drug Targets CNS Neurol Disord, 2005. 4(1): p. 41-50; hereby incorporated by reference in its entirety). HATs, however, have been investigated to a lesser extent. HAT activators have been reported, but many are neither soluble nor membrane permeant, which makes them poor candidates for therapeutics. CTPB and CTB are HAT activators that are insoluble and membrane-impermeable (J Phys Chem B, 2007. 111(17): p. 4527-34; J Biol Chem, 2003. 278(21): p. 19134-40; each hereby incorporated by reference in its entirety). Nemorosone is another HAT activator (Chembiochem. 11(6): p. 818-27; hereby incorporated by reference in its entirety). However, these compounds suffer from unfavorable physicochemical characteristics for use in CNS diseases.

There is a need for novel HAT activators. There is also a need for novel treatments for a variety of disease states for which HAT activity is implicated. There is a further need for novel and effective treatments for neurodegenerative diseases, neurological disorders and cancers. In particular, there is a continuing need for treatment of dementia and memory loss associated with Alzheimer's disease. There is also a continuing need for treatment of cancer.

### SUMMARY

The present disclosure is directed to compounds and compositions that modulate HAT activity and their methods of use in treating a neurodegenerative disease or cancer. In various embodiments, compounds that modulate HAT activity can be HAT activators or HAT inhibitors. Thus, pharmaceutical compositions may comprise a HAT modulating compound, and the methods may comprise administering to a subject a compound or composition that modulates HAT activity.

In some embodiments, the present disclosure provides a compound of Formula (I),

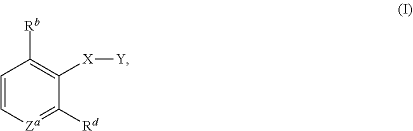

wherein,
X is —C(O)N(R^{a1})— or —N(R^{a2})C(O)—;
Y is —C₁₋₆-alkyl,

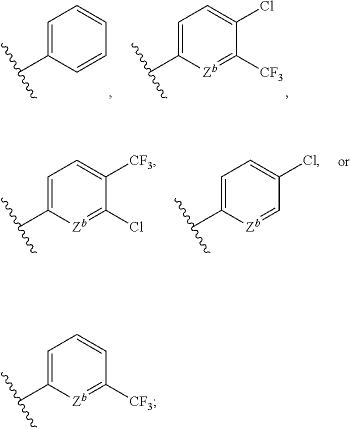

Z^{a} and Z^{b} are each independently CH or N;
R^{a1} and R^{a2} are each independently H, —C₁₋₃ alkyl, —(CH₂)ₘ—R^{c};
R^{b} is H, halogen, —OH, —O—C₁₋₆-alkyl;
R^{c} is —OH, —O-alkyl, —NH(C₁₋₃-alkyl), or —N(C₁₋₃-alkyl)₂;
R^{d} is —OH, —OMe, —OEt, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3,
   with the proviso that
   when X is —C(O)N(H)—, Y is

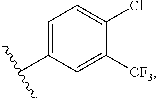 [SMILES: *c1ccc(Cl)c(C(F)(F)F)c1]

Z^{a} is —CH and R^{b} is —OMe or —OEt; R^{d} is not —OH, —O—(CH₂)₂—NMe₂, —O—(CH₂)₂—NEt₂, —O—(CH₂)₃—NMe₂, —O—(CH₂)₃—NEt₂, or —N(H)—(CH₂)₂—NMe₂;
when X is —C(O)N(H)—, Y is

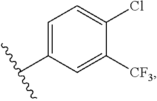 [SMILES: *c1ccc(Cl)c(C(F)(F)F)c1]

Z^{a} is —CH and R^{b} is H; R^{d} is not —OH, —OMe, —OEt, —O—(CH₂)₂—NMe₂ or —N(H)—(CH₂)₂—NMe₂;
when X is —C(O)N(Me)-, Y is

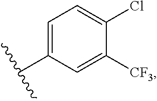 [SMILES: *c1ccc(Cl)c(C(F)(F)F)c1]

Z^{a} is —CH and R^{b} is —OEt; R^{d} is not —O—(CH₂)₂—NMe₂; and
when X is —C(O)N(Me)-, Y is

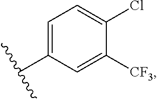 [SMILES: *c1ccc(Cl)c(C(F)(F)F)c1]

Z^{a} is —CH and R^{b} is —OH; R^{d} is not —OH.

In some embodiments, the pharmaceutical compositions disclosed herein comprise a compound of Formula (I) and a pharmaceutically acceptable excipient. In certain embodiments, the compound of Formula (I) is a HAT activator. In other embodiments, the compound of Formula (I) is a HAT inhibitor.

In some embodiments, the present disclosure provides a method of increasing histone acetylation in a subject, the method comprising administering to the subject a therapeutically effective amount of a compound of Formula (I) or a composition comprising a compound of Formula (I).

In some embodiments, the present disclosure provides a method of treating a neurodegenerative disease or condition in a subject in need thereof, the method comprising administering to the subject a therapeutically effective amount of a compound of Formula (I) or a composition comprising a compound of Formula (I).

In some embodiments, the present disclosure provides a method of treating cancer in a subject in need thereof, the method comprising administering to the subject a therapeutically effective amount of a compound of Formula (I) or a composition comprising a compound of Formula (I).

### BRIEF DESCRIPTION OF THE FIGURES

FIG. 1A and FIG. 1B provide graphs of the average values of lysine residue acetylation and standard error ranges for RA010115.

FIG. 2 provides a graph of the average values of lysine residue acetylation and standard error ranges for RA010143.

FIG. 3A and FIG. 3B provide graphs of the average values of lysine residue acetylation and standard error ranges for RA010146.

FIG. 4A and FIG. 4B provide graphs of the average values of lysine residue acetylation and standard error ranges for RA010155.

FIG. 5A and FIG. 5B provide graphs of the average values of lysine residue acetylation and standard error ranges for RA010159.

FIG. 6A and FIG. 6B provide graphs of the average values of lysine residue acetylation and standard error ranges for RA010160.

FIG. 7A and FIG. 7B provide graphs of the average values of lysine residue acetylation and standard error ranges for RA010162.

FIG. 8A and FIG. 8B provide graphs of the average values of lysine residue acetylation and standard error ranges for RA010163.

FIG. 9A and FIG. 9B provide graphs of the average values of lysine residue acetylation and standard error ranges for RA010165.

FIG. 10A and FIG. 10B provide graphs of the average values of lysine residue acetylation and standard error ranges for RA010168.

FIG. 11A and FIG. 11B provide graphs of the average values of lysine residue acetylation and standard error ranges for RA010171.

FIG. 12A and FIG. 12B provide graphs of the average values of lysine residue acetylation and standard error ranges for RA010900 in DMSO.

FIG. 13A and FIG. 13B provide graphs of the average values of lysine residue acetylation and standard error ranges for RA010900 in water.

FIG. 14A and FIG. 14B provide graphs of the average values of lysine residue acetylation and standard error ranges for RA013005.

FIG. 15 provides a graph of the average values of lysine residue acetylation and standard error ranges for RA013011.

FIG. 16A and FIG. 16B provide graphs of the average values of lysine residue acetylation and standard error ranges for RA013012.

FIG. 17A and FIG. 17B provide graphs of the average values of lysine residue acetylation and standard error ranges for RA013886.

FIG. 18A and FIG. 18B provide graphs of the average values of lysine residue acetylation and standard error ranges for RA013894.

FIG. 19A and FIG. 19B provide graphs of the average values of lysine residue acetylation and standard error ranges for RA013905.

FIG. 20A and FIG. 20B provide graphs of the average values of lysine residue acetylation and standard error ranges for RA013915.

FIG. 21A and FIG. 21B provide graphs of the average values of lysine residue acetylation and standard error ranges for RA013917.

FIG. 22A and FIG. 22B provide graphs of the average values of lysine residue acetylation and standard error ranges for RA013919.

FIG. 23A and FIG. 23B provide graphs of the average values of lysine residue acetylation and standard error ranges for RA013928.

FIG. 24A and FIG. 24B provide graphs of the average values of lysine residue acetylation and standard error ranges for RA013931.

FIG. 25A and FIG. 25B provide graphs of the average values of lysine residue acetylation and standard error ranges for RA013938.

FIG. 26 is a graph showing that RA010115 rescues oligomeric Tau (oTAU)- and Aβ (oAβ)-induced LTP deficits.

FIG. 27 is a graph showing that RA010115 rescues oTau- and oAβ-induced defects in the 2 day radial arm water maze test of spatial short-term memory.

FIG. 28 is a graph showing that RA010115 rescues oTau- and oAβ-induced defects in contextual fear memory.

FIG. 29 shows a graph with the average freezing in cued fear associative memory test in the presence oTau and oAβ with and without RA010115.

FIG. 30A and FIG. 30B show graphs with the average time and speed to reach a platform located above the surface of the water in the presence oTau and oAβ with and without RA010115.

FIG. 31A and FIG. 31B show the performance of mice in the open field test in the presence oTau and oAβ with and without RA010115. Both the time spent in the center of the arena (A) and the number of entries in the center (B) are plot.

FIG. 32 shows that the sensory threshold is not affected by the presence oTau and oAβ with and without RA010115.

FIG. 33 is a graph showing that RA013915 rescues oligomeric Tau (oTAU)- and (oAβ)-induced LTP deficits.

### DETAILED DESCRIPTION

In the following description, certain specific details are set forth in order to provide a thorough understanding of various embodiments. However, one skilled in the art will understand that the invention can be practiced without these details. In other instances, well-known structures have not been shown or described in detail to avoid unnecessarily obscuring descriptions of the embodiments.

All definitions, as defined and used herein, should be understood to control over dictionary definitions, definitions in documents incorporated by reference, and/or ordinary meanings of the defined terms. Use of flow diagrams is not meant to be limiting with respect to the order of operations performed for all embodiments. The indefinite articles “a” and “an,” as used herein in the specification and in the claims, unless clearly indicated to the contrary, should be understood to mean “at least one.”

Reference throughout this specification to “one embodiment” or “an embodiment,” etc. means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

“Alkyl” or “alkyl group” refers to a fully saturated, straight or branched hydrocarbon chain radical, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical. Alkylenes comprising any number of carbon atoms from 1 to 12 are included. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Examples of C₁-C₃ alkyl includes methyl, ethyl, n-propyl, and i-propyl. Examples of C₁-C₄ alkyl includes methyl, ethyl, n-propyl, i-propyl, n-butyl, i-butyl, and sec-butyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl groups comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbons in Rₐ, as defined above. For example, “C1-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

“Aryl” refers to a hydrocarbon ring system radical comprising hydrogen, 5 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” refers to a radical of the formula —R_{b}—R_{c} where R_{b} is an alkylene, alkenylene or alkynylene group as defined above and R^{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above that is substituted by one or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” refers to a radical of the formula —R_{b}—Rₑ where R_{b} is an alkylene, alkenylene, or alkynylene chain as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in this disclosure, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

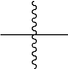

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

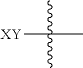

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

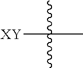

infers that when R³ is “XY”, the point of attachment bond is the same bond as the bond by which R³ is depicted as being bonded to CH₃.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

The compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

A “tautomer” refers to a proton shift from one atom of a molecule to another atom of the same molecule. The present invention includes tautomers of any said compounds.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, a “subject” can be a human, non-human primate, mammal, rat, mouse, cow, horse, pig, sheep, goat, dog, cat, insect and the like. The subject can be suspected of having or at risk for having a cancer, such as a blood cancer, or another disease or condition. Diagnostic methods for various cancers, and the clinical delineation of cancer, are known to those of ordinary skill in the art. The subject can also be suspected of having an infection or abnormal cardiovascular function.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

“Treating” or “treatment” as used herein covers the treatment of the disease or condition of interest in a mammal, preferably a human, having the disease or condition of interest, and includes (but is not limited to):
1. preventing the disease or condition from occurring in a mammal, in particular, when such mammal is predisposed to the condition but has not yet been diagnosed as having it;
2. inhibiting the disease or condition, i.e., arresting its development;
3. relieving the disease or condition, i.e., causing regression of the disease or condition (ranging from reducing the severity of the disease or condition to curing the disease of condition); or
4. relieving the symptoms resulting from the disease or condition, i.e., relieving pain without addressing the underlying disease or condition. As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

Throughout the present specification, the terms “about” and/or “approximately” can be used in conjunction with numerical values and/or ranges. The term “about” is understood to mean those values near to a recited value. For example, “about 40 [units]” can mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values herein. Furthermore, the phrases “less than about [a value]” or “greater than about [a value]” should be understood in view of the definition of the term “about” provided herein. The terms “about” and “approximately” can be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

Following below are more detailed descriptions of various concepts related to, and embodiments of inventive compounds and methods for the treatment of cancer and neurodegenerative diseases. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

### Compounds

In some embodiments of the present disclosure, compounds of Formula (I) are provided

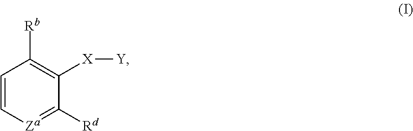

wherein
X is —C(O)N(R^{a1})— or —N(R^{a2})C(O)—;
Y is —C₁₋₆-alkyl,

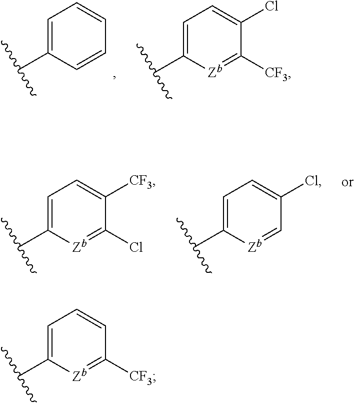

Z^{a} and Z^{b} are each independently CH or N;
R^{a1} and R^{a2} are each independently H, —C₁₋₃ alkyl, —(CH₂)ₘ—R^{c};
R^{b} is H, halogen, —OH, —O—C₁₋₆-alkyl;
R^{c} is —OH, —O-alkyl, —NH(C₁₋₃-alkyl), or —N(C₁₋₃-alkyl)₂;
R^{d} is —OH, —OMe, —OEt, —OPr, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3,
   with the proviso that
   when X is —C(O)N(H)—, Y is

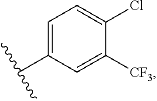 [SMILES: *c1ccc(Cl)c(C(F)(F)F)c1]

Z^{a} is —CH and R^{b} is —OMe or —OEt; R^{d} is not —OH, —O—(CH₂)₂—NMe₂, —O—(CH₂)₂—NEt₂, —O—(CH₂)₃—NMe₂, —O—(CH₂)₃—NEt₂, or —N(H)—(CH₂)₂—NMe₂;
when X is —C(O)N(H)—, Y is

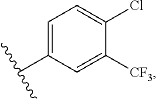 [SMILES: *c1ccc(Cl)c(C(F)(F)F)c1]

Z^{a} is —CH and R^{b} is H; R^{d} is not —OH, —OMe, —OEt, —O—(CH₂)₂—NMe₂ or —N(H)—(CH₂)₂—NMe₂;
when X is —C(O)N(Me)-, Y is

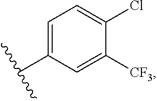 [SMILES: *c1ccc(Cl)c(C(F)(F)F)c1]

Z^{a} is —CH and R_{b} is —OEt; R_{d} is not —O—(CH₂)₂—NMe₂; and
when X is —C(O)N(Me)-, Y is

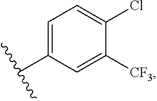 [SMILES: *c1ccc(Cl)c(C(F)(F)F)c1]

Z^{a} is —CH and R^{b} is —OH; R^{d} is not —OH.

In some embodiments, Formula (I) excludes compounds having the structures:

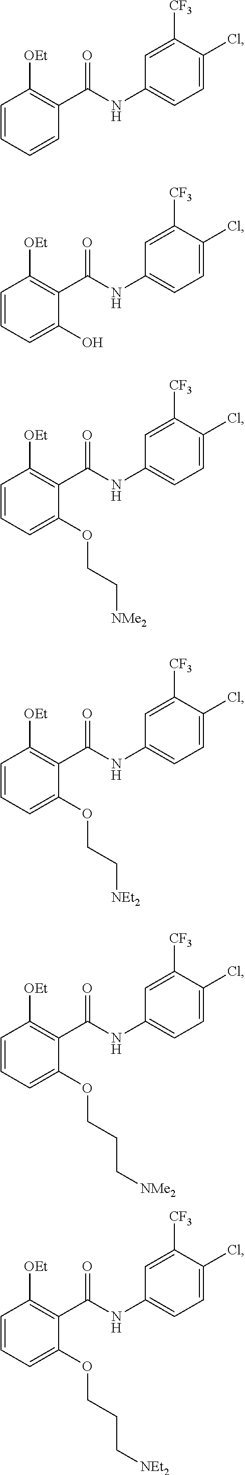

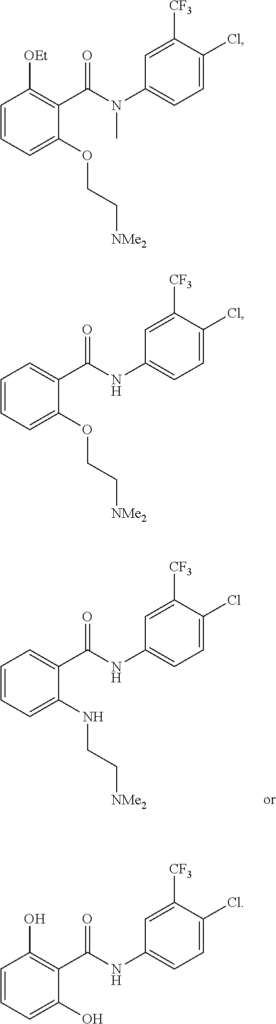 [SMILES: CCOc1cccc(OCCN(C)C)c1C(=O)N(C)c1ccc(Cl)c(C(F)(F)F)c1 ; CN(C)CCOc1ccccc1C(=O)Nc1ccc(Cl)c(C(F)(F)F)c1 ; CN(C)CCNc1ccccc1C(=O)Nc1ccc(Cl)c(C(F)(F)F)c1 ; O=C(Nc1ccc(Cl)c(C(F)(F)F)c1)c1c(O)cccc1O]

In some embodiments, the present disclosure provides compounds of Formula (I), wherein
X is —C(O)N(R^{a1})— or —N(R^{a2})C(O)—;
Y is —C₁₋₆-alkyl,

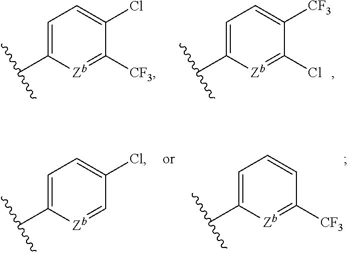

Z^{a} and Z^{b} are each independently CH or N;
R^{a1} and R^{a2} are each independently H, —C₁₋₃-alkyl, —(CH₂)ₘ—R^{c};
R^{b} is H, halogen, —OH, —OMe, —OEt, —OPr, —OiPr, or OBu;
R^{c} is —OH, —O-alkyl, or —N(C₁₋₃ alkyl)₂;
R^{d} is —OH, —OMe, —OEt, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3.

In some embodiments, the present disclosure provides compounds of Formula (I), wherein

X is —C(O)N(R^{a1})— or —N(R^{a2})C(O)—;
Y is Me,

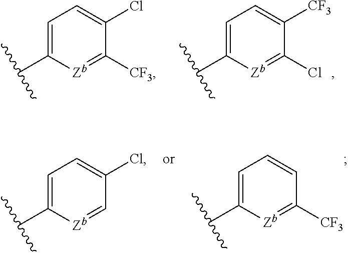

Z^{a} and Z^{b} are each independently CH or N;
R^{a1} and R^{a2} are each independently H, —C₁₋₃-alkyl, —(CH₂)ₘ—R^{c};
R^{b} is H, halogen, —OH, —OMe, —OEt, —OPr, —OiPr, or OBu;
R^{c} is —OH, —O-alkyl, or —N(C₁₋₃ alkyl)₂;
R^{d} is —OH, —OMe, —OEt, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3.

In some embodiments of Formula (I), X is —C(O)N(R^{a1})—. In other embodiments, X is —N(R^{a2})C(O)—.

In some embodiments of Formula (I), Y is —C₁₋₆-alkyl,

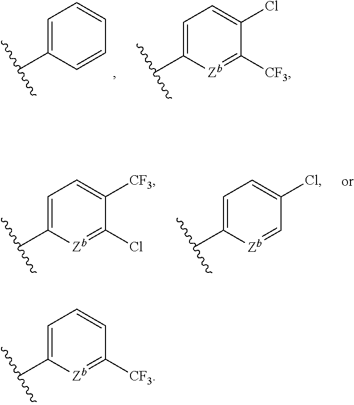

In other embodiments, Y is —C₁₋₆-alkyl,

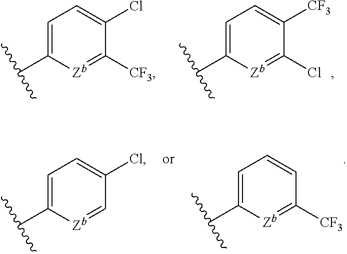

In still other embodiments, Y is —C₁₋₆-alkyl,

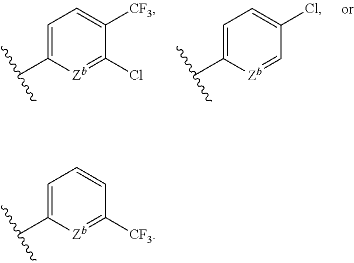

In yet other embodiments, Y is

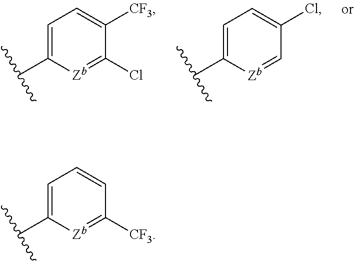

In another embodiment, Y is,

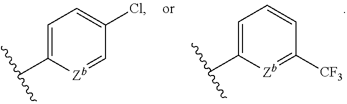

In certain embodiments, Y is

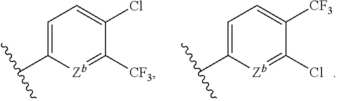

In specific embodiments, Y is

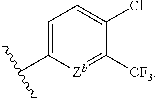

In other specific embodiments, Y is

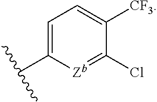

In still other specific embodiments, Y is

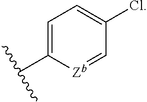

In yet other specific embodiments, Y is

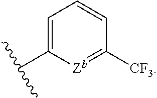

In some embodiments, Y is —C₁₋₆-alkyl. In some embodiments, the —C₁₋₆-alkyl is methyl, ethyl, propyl, isopropyl, butyl, pentyl, or hexyl. In other embodiments, the —C₁₋₆-alkyl is methyl, ethyl or propyl. In certain embodiments, the —C₁₋₆-alkyl is methyl or ethyl. In specific embodiments, the —C₁₋₆-alkyl is methyl.

In some embodiments of Formula (I), Z^{a} and Z^{b} are CH. In some embodiments, Z^{a} is N and Z^{b} is N. In other embodiments, Z^{a} is N and Z^{b} is CH. In still other embodiments, Z^{a} is CH and Z^{b} is N.

In some embodiments of Formula (I), R^{a1} and R^{a2} are each independently H, —C₁₋₃-alkyl, —(CH₂)ₘ—R^{c}. In other embodiments, R^{a1} and R^{a2} are each independently H or —C₁₋₃-alkyl. In certain embodiments, R^{a1} and R^{a2} are each independently H. In certain other embodiments, R^{a1} and R^{a2} are each independently —C₁₋₃-alkyl. In some embodiments, the —C₁₋₃-alkyl is methyl, ethyl, or propyl. In other embodiments, the —C₁₋₃-alkyl is methyl or ethyl. In specific embodiments, the —C₁₋₃-alkyl is methyl. In some embodiments, R^{a1} and R^{a2} are each independently —CH₂)ₘ—R^{c}.

In some embodiments of Formula (I), R^{b} is halogen, —OH, or —O—C₁₋₃-alkyl. In other embodiments, R^{b} is halogen or —O—C₁₋₃-alkyl. In still other embodiments, R^{b} is —O—C₁₋₃-alkyl. In some embodiments, the —O—C₁₋₃-alkyl is selected from the group consisting of —OMe, —OEt, —OPr, or —OiPr. In other embodiments, the —O—C₁₋₃-alkyl is selected from the group consisting of —OMe, —OPr, or —OiPr.

In some embodiments of Formula (I), R^{c} is —OH, —O-alkyl, —NH(C₁₋₃-alkyl), or —N(C₁₋₃-alkyl)₂. In other embodiments, R^{c} is —OH or —O-alkyl. In still other embodiments, R^{c} is —NH(C₁₋₃-alkyl), or —N(C₁₋₃-alkyl)₂. In yet other embodiments, R^{c} is —N(C₁₋₃-alkyl)₂. In some embodiments, the C₁₋₃-alkyl is selected from the group consistent of methyl, ethyl, or propyl. In other embodiments, the C₁₋₃-alkyl is methyl or ethyl. In specific embodiments, the C₁₋₃-alkyl is methyl.

In some embodiments of Formula (I), R^{d} is —OH, —OMe, —OEt, —O—(CH₂)ₙ—R^{e1}, —N(H)—(CH₂)ₙ—R^{e2}; or —N(Me)-(CH₂)ₙ—R^{e2}. In other embodiments, R^{d} is —OMe, —OEt, —O—(CH₂)ₙ—R^{e1}, —N(H)—(CH₂)ₙ—R^{e2}; or —N(Me)-(CH₂)ₙ—R^{e2}. In still other embodiments, R^{d} is —OMe, —OEt, or —O—(CH₂)ₙ—R^{e1}. In yet other embodiments, —O—(CH₂)ₙ—R^{e1}, —N(H)—(CH₂)ₙ—R^{e2}; or —N(Me)-(CH₂)ₙ—R^{e2}. In certain embodiments, R^{d} is —OEt, —O—(CH₂)ₙ—R^{e1}, —N(H)—(CH₂)ₙ—R^{e2}; or —N(Me)-(CH₂)ₙ—R^{e2}. In other certain embodiments, R^{d}—OMe, —O—(CH₂)ₙ—R^{e1}, —N(H)—(CH₂)ₙ—R^{e2}; or —N(Me)-(CH₂)ₙ—R^{e2}.

In some embodiments of Formula (I), R^{e1} and R^{e2} are each independently —OH or —OMe. In other embodiments, R^{e1} and R^{e2} are each independently —NH₂, —NHMe, —NMe₂, —NHEt, or —NEt₂. In still other embodiments, R^{e1} and R^{e2} are each independently —NH₂, —NHMe, or —NMe₂. In certain embodiments, R^{e1} and R^{e2} are each independently —NMe₂ or —NEt₂. In specific embodiments, R^{e1} and R^{e2} are each independently —NMe₂.

In some embodiments of Formula (I), m is 2 or 3. In other embodiments, m is 2. In certain embodiments, m is 3.

In some embodiments of Formula (I), n is 2. In other embodiments, n is 3.

In some embodiments of Formula (I), X is —C(O)N(R^{a1})— and R^{a1} is H or —C₁₋₃-alkyl. In other embodiments, X is —C(O)N(R^{a1})— and R^{a1} is H or Me. In still other embodiments, X is —C(O)N(R^{a1})— and R^{a1} is H. In yet other embodiments, X is —C(O)N(R^{a1})— and R^{a1} is —C₁₋₃-alkyl. In another embodiment, X is —C(O)N(R^{a1})— and R^{a1} is Me.

In some embodiments of Formula (I), X is —N(R^{a2})C(O)— and R^{a2} is H or —C₁₋₃-alkyl. In other embodiments, X is —N(R^{a2})C(O)— and R^{a2} is H or Me. In still other embodiments, X is —N(R^{a2})C(O)— and R^{a2} is H. In yet other embodiments, X is —N(R^{a2})C(O)— and R^{a2} is —C₁₋₃-alkyl. In another embodiment, X is —N(R^{a2})C(O)— and R^{a2} is Me.

In some embodiments of Formula (I), the compounds have a structure as in Table 1, below, or a pharmaceutically acceptable salt or solvate thereof.

TABLE 1

| | |
|---|---|
| 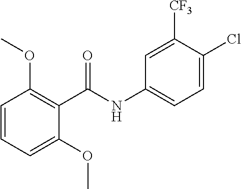 [SMILES: COc1cccc(OC)c1C(=O)Nc1ccc(Cl)c(C(F)(F)F)c1] | RA010115 |
| 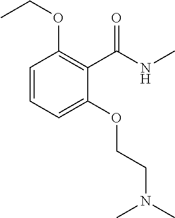 [SMILES: CCOc1cccc(OCCN(C)C)c1C(=O)NC] | RA010154 |
| 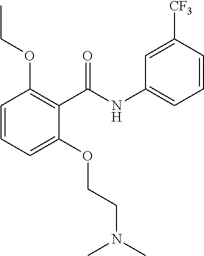 [SMILES: CCOc1cccc(OCCN(C)C)c1C(=O)Nc1cccc(C(F)(F)F)c1] | RA013928 |
| 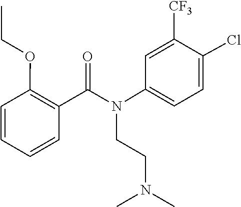 [SMILES: CCOc1ccccc1C(=O)N(CCN(C)C)c1ccc(Cl)c(C(F)(F)F)c1] | RA013886 |
| 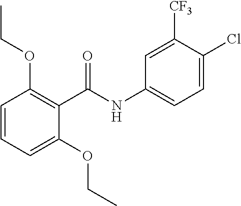 [SMILES: CCOc1cccc(OCC)c1C(=O)Nc1ccc(Cl)c(C(F)(F)F)c1] | RA010171 |
| 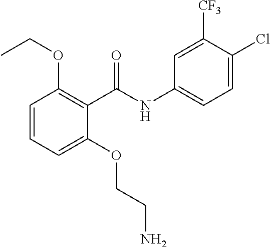 [SMILES: CCOc1cccc(OCCN)c1C(=O)Nc1ccc(Cl)c(C(F)(F)F)c1] | RA013895 |
| 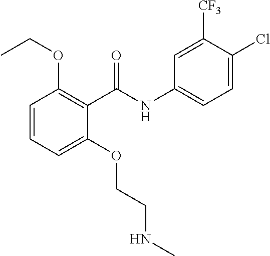 [SMILES: CCOc1cccc(OCCNC)c1C(=O)Nc1ccc(Cl)c(C(F)(F)F)c1] | RA013894 |
| 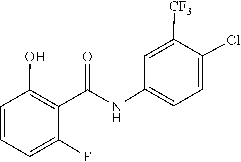 [SMILES: O=C(Nc1ccc(Cl)c(C(F)(F)F)c1)c1c(O)cccc1F] | RA010158 |
| 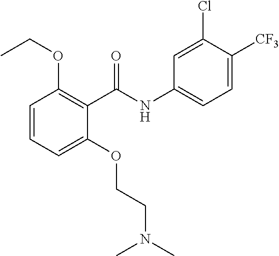 [SMILES: CCOc1cccc(OCCN(C)C)c1C(=O)Nc1ccc(C(F)(F)F)c(Cl)c1] | RA013911 |
| 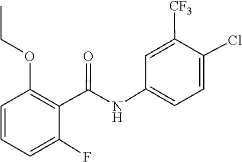 [SMILES: CCOc1cccc(F)c1C(=O)Nc1ccc(Cl)c(C(F)(F)F)c1] | RA010156 |
| 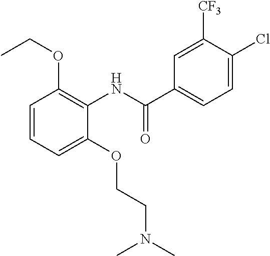 [SMILES: CCOc1cccc(OCCN(C)C)c1NC(=O)c1ccc(Cl)c(C(F)(F)F)c1] | RA013919 |
| 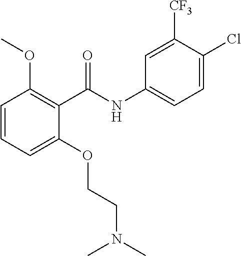 [SMILES: COc1cccc(OCCN(C)C)c1C(=O)Nc1ccc(Cl)c(C(F)(F)F)c1] | RA010900 |
| 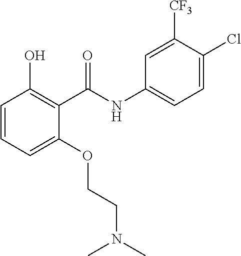 [SMILES: CN(C)CCOc1cccc(O)c1C(=O)Nc1ccc(Cl)c(C(F)(F)F)c1] | RA013905 |
| 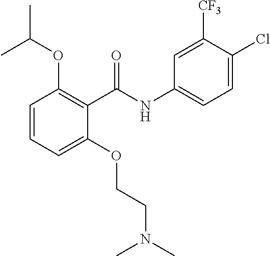 [SMILES: CC(C)Oc1cccc(OCCN(C)C)c1C(=O)Nc1ccc(Cl)c(C(F)(F)F)c1] | RA010143 |
| 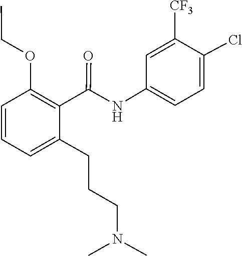 [SMILES: CCOc1cccc(CCCN(C)C)c1C(=O)Nc1ccc(Cl)c(C(F)(F)F)c1] | |
| 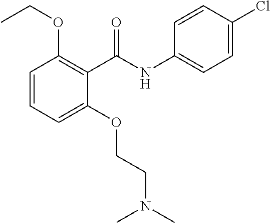 [SMILES: CCOc1cccc(OCCN(C)C)c1C(=O)Nc1ccc(Cl)cc1] | RA010150 |
| 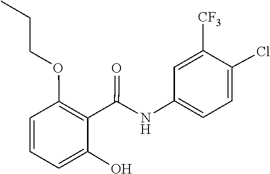 [SMILES: CCCOc1cccc(O)c1C(=O)Nc1ccc(Cl)c(C(F)(F)F)c1] | RA013931 |
| 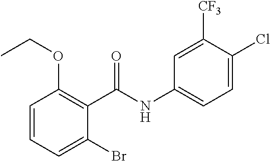 [SMILES: CCOc1cccc(Br)c1C(=O)Nc1ccc(Cl)c(C(F)(F)F)c1] | RA010148 |
| 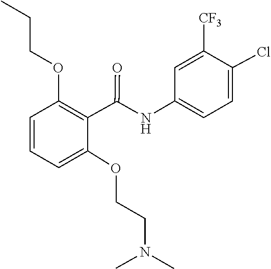 [SMILES: CCCOc1cccc(OCCN(C)C)c1C(=O)Nc1ccc(Cl)c(C(F)(F)F)c1] | RA013938 |
| 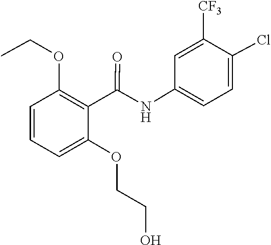 [SMILES: CCOc1cccc(OCCO)c1C(=O)Nc1ccc(Cl)c(C(F)(F)F)c1] | RA010146 |
| 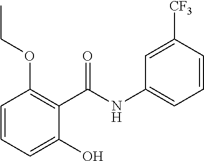 [SMILES: CCOc1cccc(O)c1C(=O)Nc1cccc(C(F)(F)F)c1] | RA010160 |
| 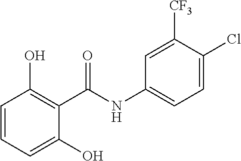 [SMILES: O=C(Nc1ccc(Cl)c(C(F)(F)F)c1)c1c(O)cccc1O] | RA013920 |
| 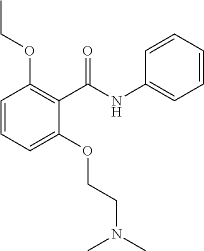 [SMILES: CCOc1cccc(OCCN(C)C)c1C(=O)Nc1ccccc1] | RA013910 |
| 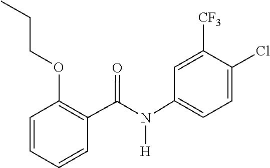 [SMILES: CCCOc1ccccc1C(=O)Nc1ccc(Cl)c(C(F)(F)F)c1] | RA013929 |
| 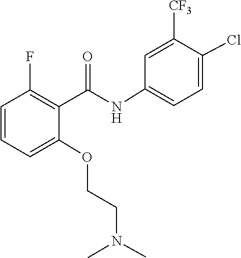 [SMILES: CN(C)CCOc1cccc(F)c1C(=O)Nc1ccc(Cl)c(C(F)(F)F)c1] | RA010162 |
| 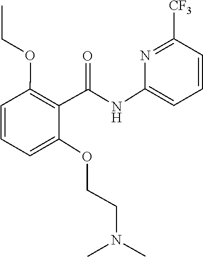 [SMILES: CCOc1cccc(OCCN(C)C)c1C(=O)Nc1cccc(C(F)(F)F)n1] | RA01311 |
| 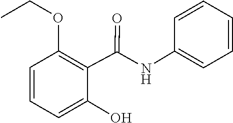 [SMILES: CCOc1cccc(O)c1C(=O)Nc1ccccc1] | RA010166 |
| 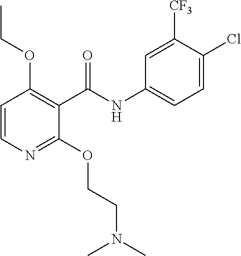 [SMILES: CCOc1ccnc(OCCN(C)C)c1C(=O)Nc1ccc(Cl)c(C(F)(F)F)c1] | RA013005 |
| 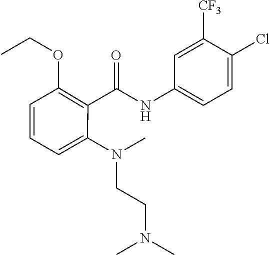 [SMILES: CCOc1cccc(N(C)CCN(C)C)c1C(=O)Nc1ccc(Cl)c(C(F)(F)F)c1] | RA010163 |
| 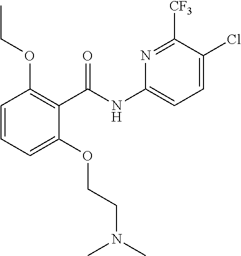 [SMILES: CCOc1cccc(OCCN(C)C)c1C(=O)Nc1ccc(Cl)c(C(F)(F)F)n1] | RA013915 |
| 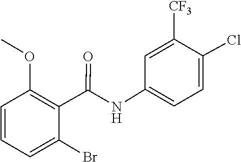 [SMILES: COc1cccc(Br)c1C(=O)Nc1ccc(Cl)c(C(F)(F)F)c1] | RA010140 |
| 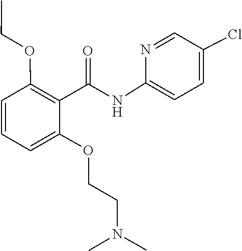 [SMILES: CCOc1cccc(OCCN(C)C)c1C(=O)Nc1ccc(Cl)cn1] | RA013917 |
| 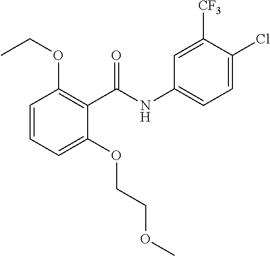 [SMILES: CCOc1cccc(OCCOC)c1C(=O)Nc1ccc(Cl)c(C(F)(F)F)c1] | RA010155 |
| 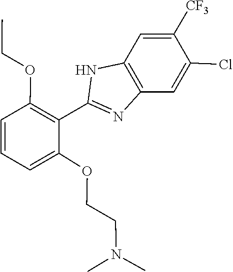 [SMILES: CCOc1cccc(OCCN(C)C)c1-c1nc2cc(Cl)c(C(F)(F)F)cc2[nH]1] | RA013012 |
| 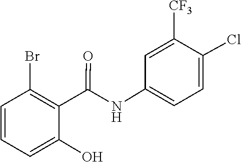 [SMILES: O=C(Nc1ccc(Cl)c(C(F)(F)F)c1)c1c(O)cccc1Br] | RA010132 |

In accordance with certain embodiments, the compound has a structure of:

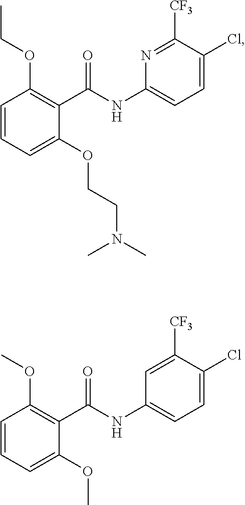 [SMILES: CCOc1cccc(OCCN(C)C)c1C(=O)Nc1ccc(Cl)c(C(F)(F)F)n1 ; COc1cccc(OC)c1C(=O)Nc1ccc(Cl)c(C(F)(F)F)c1]

or a pharmaceutically acceptable salt or solvate thereof

### Methods of Use

In some embodiments, a HAT modulator compound can be used in combination with one or more HDAC modulators to treat a neurodegenerative disease in a subject in need thereof. In other embodiments, a HAT activator compound can be used in combination with one or more HDAC inhibitors to treat a neurodegenerative disease in a subject in need Non-limiting examples of neurodegenerative diseases include Adrenoleukodystrophy (ALD), Alcoholism, Alexander's disease, Alper's disease, Alzheimer's disease, argyrophilic grain disease (AGD), and globular glial tauopathy (GGT), the neurofibrillary tangle-predominant senile dementia (now included also in the category of primary age-related tauopathy, PART), Behavioral variant frontotemporal dementia; Semantic variant primary progressive aphasia, non-fluent/agrammatic variant primary progressive aphasia, logopenic variant primary progressive aphasia, Amyotrophic lateral sclerosis (Lou Gehrig's Disease), Ataxia telangiectasia, Batten disease (also known as Spielmeyer-Vogt-Sjögren-Batten disease), Bovine spongiform encephalopathy (BSE), Canavan disease, Cockayne syndrome, Corticobasal degeneration, Creutzfeldt-Jakob disease, Familial fatal insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases Progressive Supranuclear Palsy, Refsum's disease, Rett's syndrome, Tau-positive FrontoTemporal dementia, Tau-negative FrontoTemporal dementia, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, and Toxic encephalopathy.

In some embodiments, the neurodegenerative disease is selected from Alzheimer's Disease, ALS, Parkinson's Disease, and Huntington's Disease. In some embodiments, the neurodegenerative disease is Alzheimer's Disease. In some embodiments, the neurodegenerative disease is Huntington's Disease.

Non-limiting examples of cancers include B cell lymphoma, colon cancer, lung cancer, renal cancer, bladder cancer, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkin's lymphoma, Hodgkin's lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma, follicular lymphoma and medullary carcinoma.

In some embodiments, the cancer is colon cancer, lung cancer, renal cancer, leukemia, CNS cancer, melanoma, ovarian cancer, breast cancer, or prostate cancer.

In some embodiments, the cancer is colon cancer, renal cancer, T cell leukemia, myeloma, leukemia, acute myeloid leukemia, acute lymphocytic leukemia, renal cell carcinoma, adenocarcinoma, glioblastoma, breast carcinoma, prostate carcinoma, or lung carcinoma.

In some embodiments, the cancer is Hodgkin's lymphoma, non-Hodgkin's lymphoma, B cell lymphoma, T cell lymphoma, or follicular lymphoma. In other embodiments, the B cell lymphoma is diffuse large B-cell lymphoma. In further embodiments, the diffuse large B-cell lymphoma is a germinal center-derived diffuse large B cell lymphoma, an activated B-cell-derived (ABC) diffuse large B-cell lymphoma, or a non-germinal center diffuse large B cell lymphoma.

Epigenetic modifications including acetylation of histones may contribute to gene expression changes important to learning and memory (Science 2010: 328(5979), 701-702; herein incorporated by reference in its entirety). Addition of acetyl groups to histones by histone acyltransferases (HAT) enhances gene expression, while their removal by histone deacetylases (HDAC) reduces gene expression. Reduction in histone acetylation has recently been linked to age-induced memory impairment and various neurodegenerative diseases (Science 2010: 328(5979), 701-702; herein incorporated by reference in its entirety). HDAC inhibitors have been shown to enhance memory in mice (Nature 459, 55-60 (7 May 2009); herein incorporated by reference in its entirety). Although clinical trials of several HDAC inhibitors are currently underway to try to prevent deacetylation, the alternative strategy of increasing histone acetylation by activating HAT has not been significantly explored. Histone acetylation is discussed in, for example, U.S. Patent Publication Nos. 2010/0166781; 2010/0144885; 2009/0076155; Neuroscience 2011, 194, 272-281; and J. Phys. Chem B 2007, 111(17), 4527-4534 (each of which herein incorporated by reference in its entirety). Further details on neurodegenerative diseases, including Alzheimer's disease, can be found in WO 2011/072243 and WO 2012/088420, each incorporated by reference herein in its entirety.

In some embodiments, the invention provides for compounds with histone acetyltransferase activity which can be used in combination with one or more HDAC modulators to treat patients with cancers or neurodegenerative diseases. In some embodiments, the compounds are HAT activators. In some embodiments, the compounds are HAT inhibitors. In some embodiments, the HDAC modulator is a HDAC activator. In some embodiments, the HDAC modulator is a HDAC inhibitor. In some embodiments, the compounds have good HAT activation potency, high selectivity, reasonable pharmacokinetics and/or good permeability across the blood-brain-barrier (BBB). In some embodiments, these compounds can be used as therapy with decreased side effects for AD patients. In some embodiments, the compounds improve cognition or memory in AD and Alzheimer's-like pathologies, as well as minimize the side effects for subjects afflicted with other neurodegenerative diseases. In some embodiments, the compounds of the invention can also be developed as anti-cancer therapies. In some embodiments, acetylation of histone proteins increases gene expression in a subject resulting in enhanced memory and cognition.

In some embodiments, the invention provides a method for reducing amyloid beta (Aβ) protein deposits in a subject in need thereof, the method comprising administering to the subject a HAT activator and a HDAC inhibitor. In some embodiments, the subject exhibits abnormally elevated levels of amyloid beta plaques. In some embodiments, the subject is afflicted with Alzheimer's disease, Lewy body dementia, inclusion body myositis, or cerebral amyloid angiopathy.

In some embodiments, the invention provides a method for reducing tau protein deposits in a subject in need thereof, the method comprising administering to the subject a HAT activator and a HDAC inhibitor. In some embodiments, the subject exhibits abnormally elevated levels of neurofibrillary tangles. In some embodiments, the subject is afflicted with Alzheimer's disease, tauopathy.

In further embodiments, the invention provides for the utilization of HAT agonists in combination with one or more HDAC modulators as memory enhancers in normal subjects (for example, a subject not afflicted with a neurodegenerative disease). In further embodiments, the invention provides for the utilization of HAT agonists in combination with one or more HDAC modulators as memory enhancers in aging subjects (for example, a subject who is >55 years old). In further embodiments, the invention provides for the utilization of HAT agonists in combination with one or more HDAC modulators as memory enhancers for other conditions associated with cognitive decrease/impairment. In some embodiments, the HDAC modulator is a HDAC activator. In some embodiments, the HDAC modulator is a HDAC inhibitor. Non-limiting examples of conditions associated with cognitive decrease/impairment include a variety of syndromes associated with mental retardation and syndromes associated with learning disabilities, Parkinson's disease, Pick's disease, argyrophilic grain disease (AGD), and globular glial tauopathy (GGT), the neurofibrillary tangle-predominant senile dementia (now included also in the category of primary age-related tauopathy, PART), Behavioral variant frontotemporal dementia; Semantic variant primary progressive aphasia, non-fluent/agrammatic variant primary progressive aphasia, logopenic variant primary progressive aphasia, a Lewy body disease, amyotrophic lateral sclerosis, Huntington's disease, Creutzfeld-Jakob disease, Down syndrome, multiple system atrophy, neuronal degeneration with brain iron accumulation type I (Hallervorden-Spatz disease), pure autonomic failure, REM sleep behavior disorder, mild cognitive impairment (MCI), cerebral amyloid angiopathy (CAA), mild cognitive deficits, aging, vascular dementias mixed with Alzheimer's disease, a neurodegenerative disease characterized by abnormal amyloid deposition, and any combination thereof.

In some embodiments, the invention provides methods for identifying a combination of one or more HAT modulators and one or more HDAC modulators that can acetylate histone proteins thus increasing gene expression in a subject resulting in enhanced memory and cognition. In some embodiments, the invention provides methods for identifying a combination of one or more HAT activators and one or more HDAC inhibitors can acetylate histone proteins thus increasing gene expression in a subject resulting in enhanced memory and cognition.

To shrink the candidate pool of HAT modulator and HDAC modulator combinations to be tested in animal models of neurodegenerative diseases, such as animals that exhibit elevated levels of inclusion bodies, for example Aβ accumulation animal models (e.g., animal models of AD), or, for example, or tau accumulation animal models (e.g. animal model of tauopathy), or a mouse model for Huntington's disease, HAT modulators or HDAC modulators can first be screened or selected based on their possession of certain characteristics, such as having one or more of: an EC₅₀ no greater than about 500 nM; a histone acetylation activity in vitro; and the ability to penetrate the BBB. HAT modulator and HDAC modulator combinations can first be screened or selected based on their possession of certain characteristics, such as having a histone acetylation activity in vitro or resulting in increased histone acetylation in vitro compared to histone acetylation in vitro of the HAT modulator or HDAC modulator alone.

In some embodiments, the candidate pool of HAT modulator and HDAC modulator combinations can be tested in animal models of neurodegenerative diseases, such as, but not limited to, animals that exhibit elevated levels of inclusion bodies, for example Aβ□ accumulation animal models (e.g., animal models of AD), or tau accumulation animal models (e.g. animal model of tauopathy), or a mouse model for Huntington's disease to determine whether they increase gene expression in a subject resulting in enhanced memory and cognition. As used herein, a HAT activator compound does not necessarily preclude the possibility that the compound may also be able to inhibit other HATs. As used herein, a HDAC inhibitor compound does not necessarily preclude the possibility that the compound may also be able to activate other HATs.

In some embodiments, the compounds of the invention are HAT modulators. The term “modulate”, as it appears herein, refers to a change in the activity or expression of a protein molecule. For example, modulation can cause an increase or a decrease in protein activity, binding characteristics, or any other biological, functional, or immunological properties of a secretase protein molecule. In some embodiments, the compounds activate HAT. In some embodiments, the compounds inhibit HAT.

In some embodiments, the compounds of the invention are HDAC modulators. The term “modulate”, as it appears herein, refers to a change in the activity or expression of a protein molecule. For example, modulation can cause an increase or a decrease in protein activity, binding characteristics, or any other biological, functional, or immunological properties of a secretase protein molecule. In some embodiments, the compounds inhibit HDAC. In some embodiments, the compounds activate HDAC.

A HAT modulator compound can be a compound that increases the activity and/or expression of a HAT molecule (e.g., p300, CBP, GCN5, GCN5L, PCAF, or HAT1) in vivo and/or in vitro. HAT modulator compounds can be compounds that exert their effect on the activity of a HAT protein via the expression, via post-translational modifications, or by other means. In some embodiments, a HAT modulator compound increases HAT protein or mRNA expression, or acetyltransferase activity by at least about 10%, at least about 20%, at least about 30%, at least about 40%, at least about 50%, at least about 60%, at least about 70%, at least about 75%, at least about 80%, at least about 85%, at least about 90%, at least about 95%, at least about 97%, at least about 99%, or 100%.

A HDAC modulator compound can be a compound that decreases the activity and/or expression of a HDAC molecule in vivo and/or in vitro. HDAC modulator compounds can be compounds that exert their effect on the activity of a HDAC protein via the expression, via post-translational modifications, or by other means. In some embodiments, a HDAC modulator compound decreases HDAC protein or mRNA expression, or deacetyltransferase activity by at least about 10%, at least about 20%, at least about 30%, at least about 40%, at least about 50%, at least about 60%, at least about 70%, at least about 75%, at least about 80%, at least about 85%, at least about 90%, at least about 95%, at least about 97%, at least about 99%, or 100%.

Test compounds or agents that bind to a HAT molecule (such as p300, CBP, GCN5, GCN5L, PCAF, or HAT1), and/or have a stimulatory effect on the activity or the expression of a HAT molecule, can be identified by various assays. The assay can be a binding assay comprising direct or indirect measurement of the binding of a test compound or a known HAT ligand to the active site of a HAT protein. The assay can also be an activity assay comprising direct or indirect measurement of the activity of a HAT molecule. The assay can also be an expression assay comprising direct or indirect measurement of the expression of a HAT mRNA or protein. The various screening assays can be combined with an in vivo assay comprising measuring the effect of the test compound on cognitive and synaptic function in an animal model for neurodegenerative disorders, such as, but not limited to, AD or Huntington's Disease. The assay can be an assay comprising measuring the effect of the test compounds on cell viability. In one embodiment, the cells are cancer cells, such as, but not limited to B-cell lymphoma cell lines, or T-cell lymphoma cell lines (e.g. Ly1, Ly7, Ly10, SU-DHL2, HH, or H9 cell lines).

The inhibitors of the expression of a HAT molecule can be identified via contacting a HAT-positive cell or tissue with a test compound and determining the expression of a HAT protein or HAT mRNA in the cell. The protein or mRNA expression level of a HAT molecule in the presence of the test compound can be compared to the protein or mRNA expression level of a HAT protein in the absence of the test compound. The test compound can then be identified as an inhibitor of expression of a HAT protein (such as p300, CBP, GCN5, GCN5L, PCAF, or HAT1) based on this comparison. In other words, the test compound can also be a HAT inhibitor compound (such as an antagonist).

Activators of the expression of a HAT molecule can also be identified via contacting a HAT-positive cell or tissue with a test compound and determining the expression of a HAT protein or HAT mRNA in the cell. The protein or mRNA expression level of a HAT molecule in the presence of the test compound can be compared to the protein or mRNA expression level of a HAT protein in the absence of the test compound. The test compound can then be identified as an activator of expression of a HAT protein (such as p300, CBP, GCN5, GCN5L, PCAF, or HAT1) based on this comparison. For example, when expression of HAT protein or mRNA is statistically or significantly more in the presence of the test compound than in its absence, the compound is identified as an activator of the expression of a HAT protein or mRNA. In other words, the test compound can also be a HAT activator compound (such as an agonist). The expression level of a HAT protein or mRNA in cells can be determined by methods described herein.

Determining the ability of a test compound to bind to a HAT molecule, a HDAC molecule or a variant thereof can be accomplished using real-time Bimolecular Interaction Analysis (BIA) [McConnell, (1992); Sjolander, S., and Urbaniczky, C. Integrated fluid handling system for biomolecular interaction analysis. Anal. Chem. 1991, 63, 2338-2345; herein incorporated by reference in its entirety]. BIA is a technology for studying biospecific interactions in real time, without labeling any of the interactants (e.g., BIA-Core™). Changes in optical phenomenon surface plasmon resonance (SPR) can be used as an indication of real-time reactions between biological molecules.

In some embodiments, the invention provides for compounds that bind to a HAT activator protein, such as p300, CBP, GCN5, GCN5L, PCAF, or HAT1. These compounds can be identified by the screening methods and assays described herein, and enhance the activity or expression of HAT activator proteins.

Test compounds or agents that bind to a HAT molecule and/or have a stimulatory effect on the activity or the expression of a HAT molecule, can be combined with one or more test compounds or agents that bind to a HDAC molecule. The assay can be an activity assay comprising direct or indirect measurement of the activity of a HAT molecule and/or a HDAC molecule. The assay can also be an expression assay comprising direct or indirect measurement of the expression of a HAT mRNA or protein and/or a HDAC mRNA or protein. The various screening assays can be combined with an in vivo assay comprising measuring the effect of a HAT activator and a HDAC inhibitor on cognitive and synaptic function in an animal model for neurodegenerative disorders, such as, but not limited to, AD or Huntington's Disease. The assay can be an assay comprising measuring the effect of the test compounds on cell viability. In one embodiment, the cells are cancer cells, such as, but not limited to B-cell lymphoma cell lines, or T-cell lymphoma cell lines. In one embodiment, the effect of a HAT activator and one or more HDAC inhibitors in combination is compared to the effect of a HAT activator or HDAC inhibitor alone.

### Pharmaceutical Compositions

In some embodiments, the present disclosure provides pharmaceutical compositions comprising an effective amount of a compound of Formula (I), or a pharmaceutically acceptable salt thereof. In some embodiments, the pharmaceutical compositions provided herein comprise one or more pharmaceutically acceptable carriers or excipients.

In various embodiments, the pharmaceutical compositions of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The effective amount of a compound of Formula (I), pharmaceutically acceptable salts, esters, prodrugs, hydrates, solvates and isomers thereof, or a pharmaceutical composition comprising a compound of Formula (I) or a pharmaceutically acceptable salt thereof may be determined by one skilled in the art based on known methods.

In one embodiment, a pharmaceutical composition or a pharmaceutical formulation of the present disclosure comprises a compound of Formula (I) or a pharmaceutically acceptable salt thereof, and a pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microtine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington's Pharmaceutical Sciences). Preferably, the composition is administered topically, instead of enterally. For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, preferably in physiologically compatible buffers such as Hank's solution, Ringer's solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Synthesis of Compounds of Formula (I)

General Synthesis Scheme for Compounds of Formula (I)

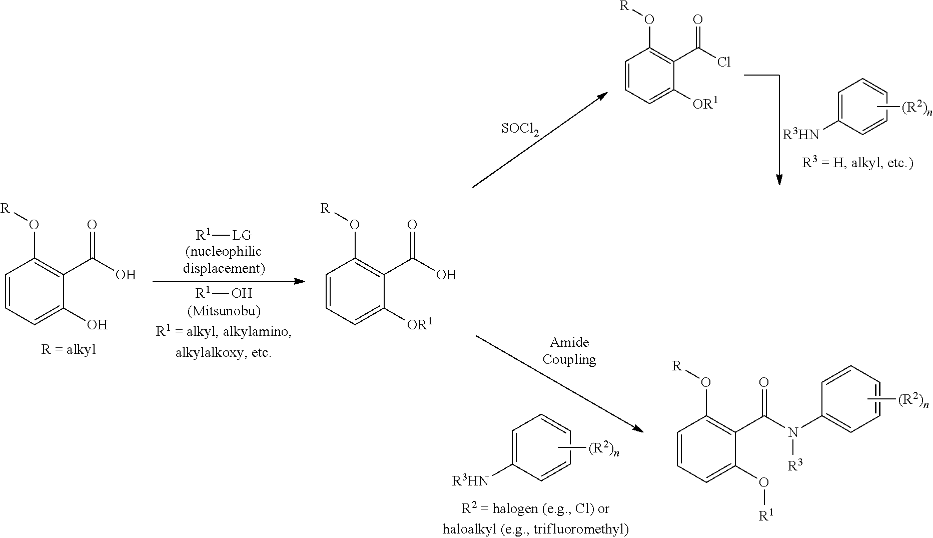

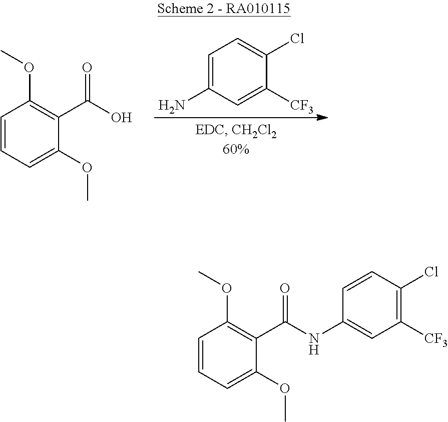 [SMILES: COc1cccc(OC)c1C(=O)O ; Nc1ccc(Cl)c(C(F)(F)F)c1 ; COc1cccc(OC)c1C(=O)Nc1ccc(Cl)c(C(F)(F)F)c1]

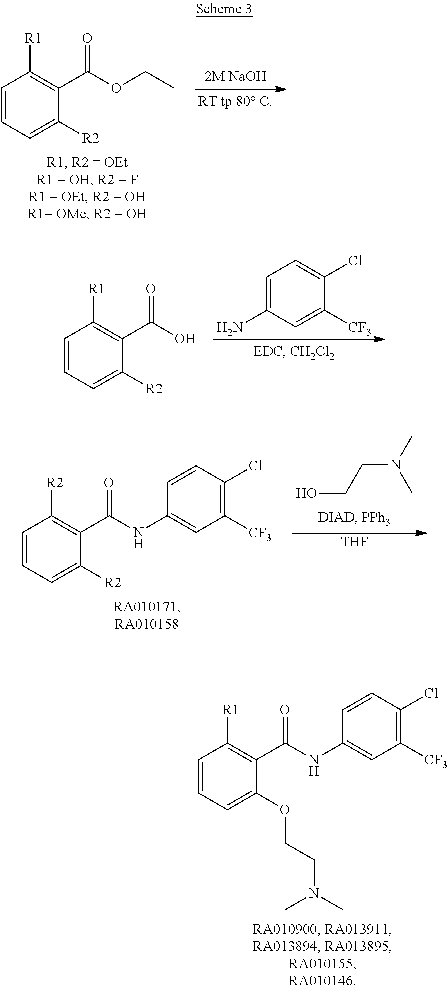

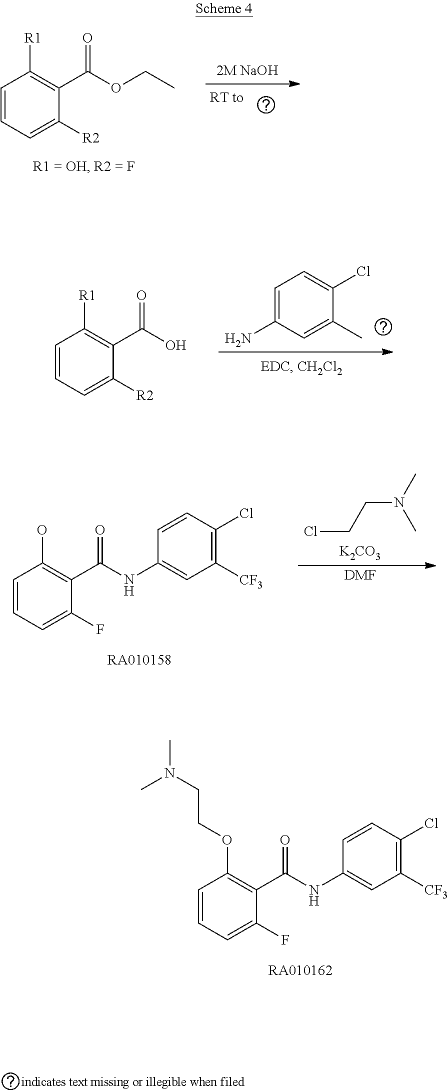

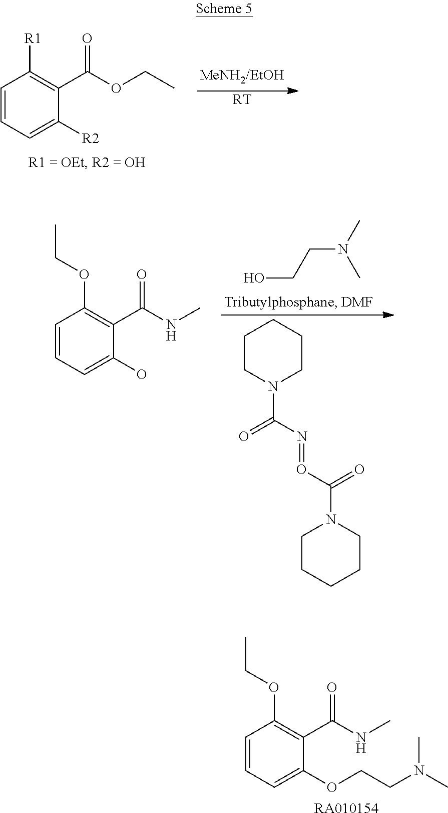

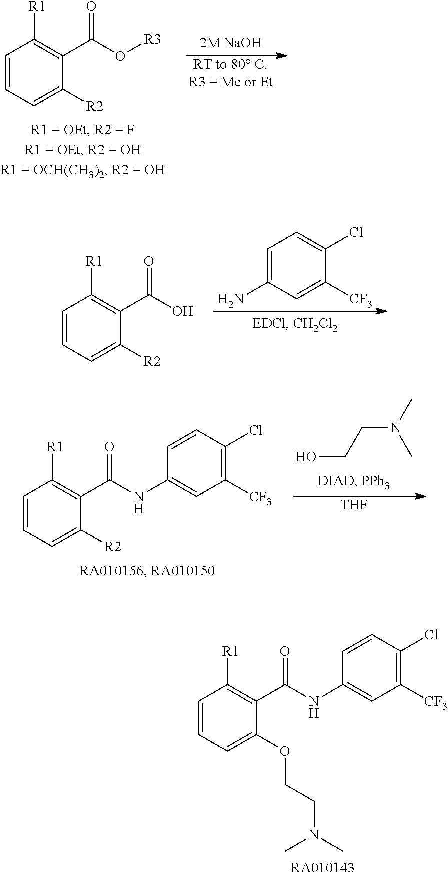

Scheme 6 was also used to synthesize the following compounds:

| Structure | Compound ID | Formulation Weight |
|---|---|---|
| 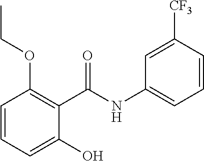 [SMILES: CCOc1cccc(O)c1C(=O)Nc1cccc(C(F)(F)F)c1] | RA010160 | 325.283 |
| 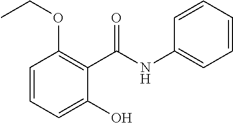 [SMILES: CCOc1cccc(O)c1C(=O)Nc1ccccc1] | RA010166 | 257.285 |
| 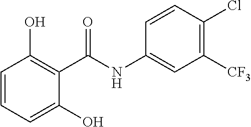 [SMILES: O=C(Nc1ccc(Cl)c(C(F)(F)F)c1)c1c(O)cccc1O] | RA013920 | 331.674 |
| 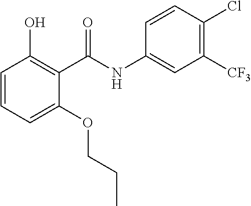 [SMILES: CCCOc1cccc(O)c1C(=O)Nc1ccc(Cl)c(C(F)(F)F)c1] | RA013931 | 373.754 |
| 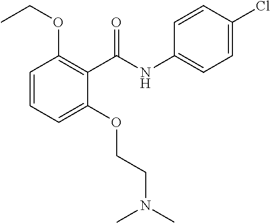 [SMILES: CCOc1cccc(OCCN(C)C)c1C(=O)Nc1ccc(Cl)cc1] | RA010150 | 399.312 |
| 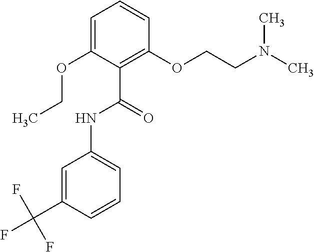 [SMILES: CCOc1cccc(OCCN(C)C)c1C(=O)Nc1cccc(C(F)(F)F)c1] | RA010168 | 432.864 |
| 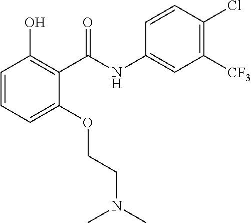 [SMILES: CN(C)CCOc1cccc(O)c1C(=O)Nc1ccc(Cl)c(C(F)(F)F)c1] | RA013905 | |
| 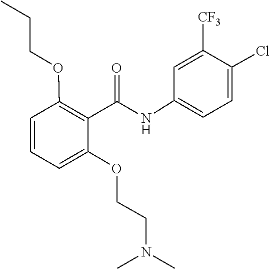 [SMILES: CCCOc1cccc(OCCN(C)C)c1C(=O)Nc1ccc(Cl)c(C(F)(F)F)c1] | RA013938 | 481.336 |

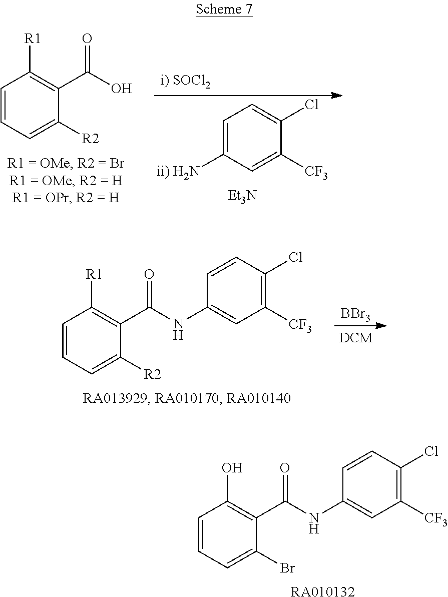

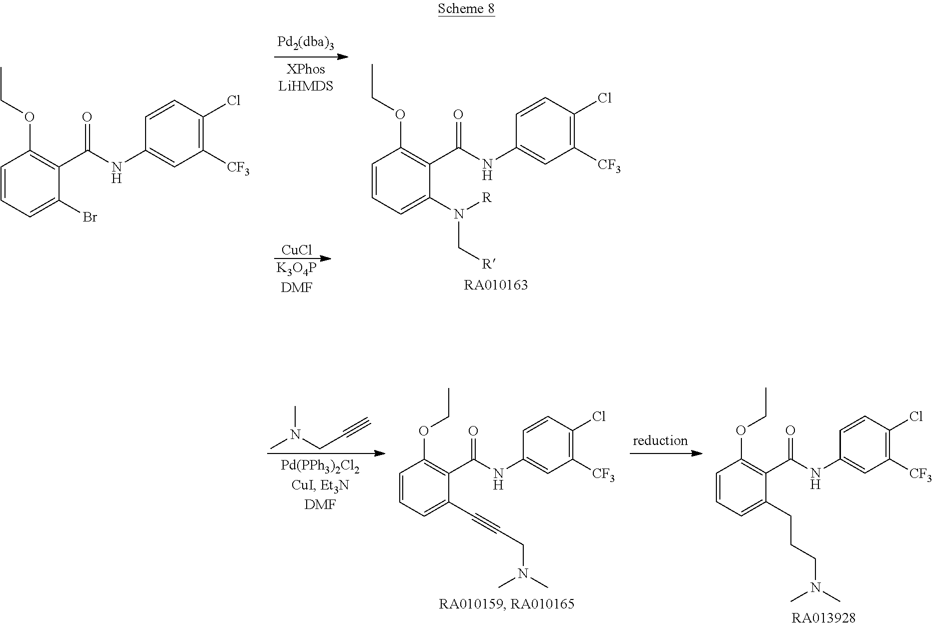

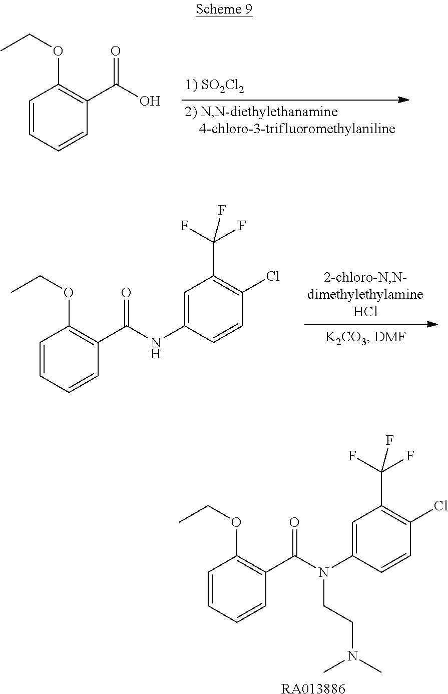

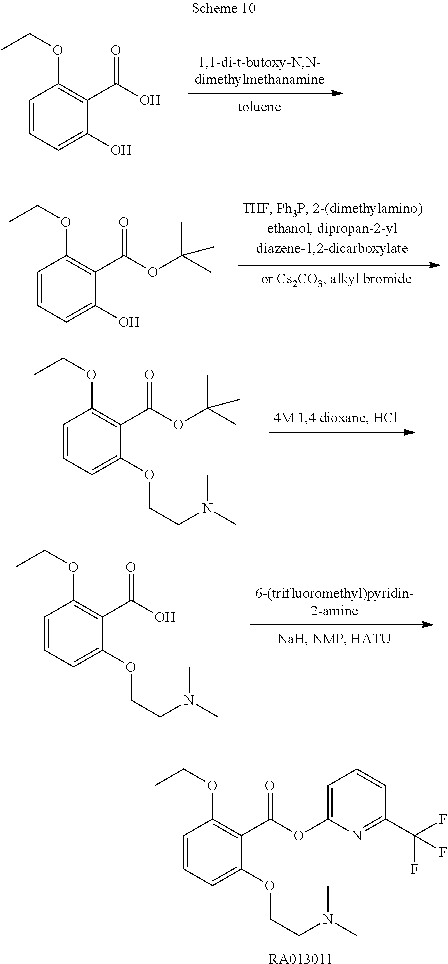

Similar schemes can be used for synthesis of RA013915

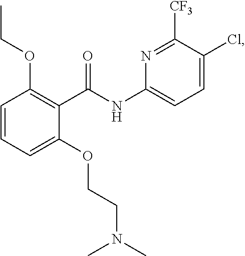 [SMILES: CCOc1cccc(OCCN(C)C)c1C(=O)Nc1ccc(Cl)c(C(F)(F)F)n1]

using thionyl chloride, N,N-diethylethanolamine, 5-chloro-6-(trifluoromethyl)pyridine-2-amine final step and RA013917

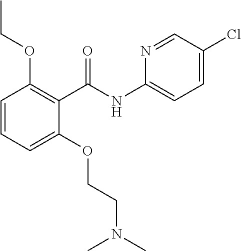 [SMILES: CCOc1cccc(OCCN(C)C)c1C(=O)Nc1ccc(Cl)cn1]

using thionyl chloride, N,N-diethylethanolamine, 5-chloropyridine-2-amine final step.

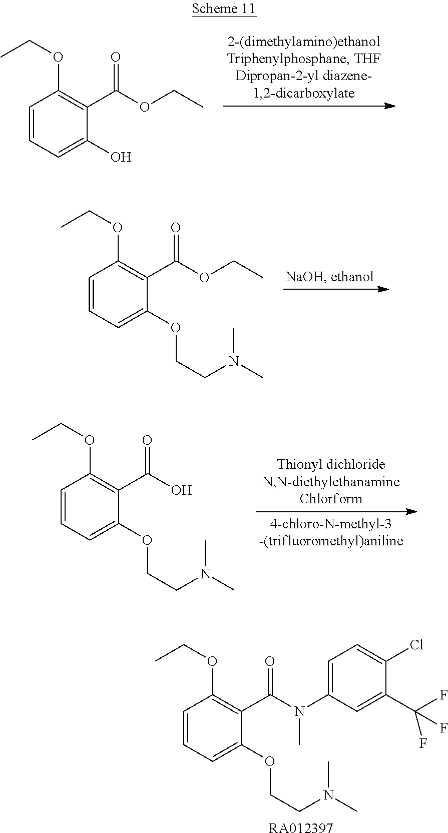

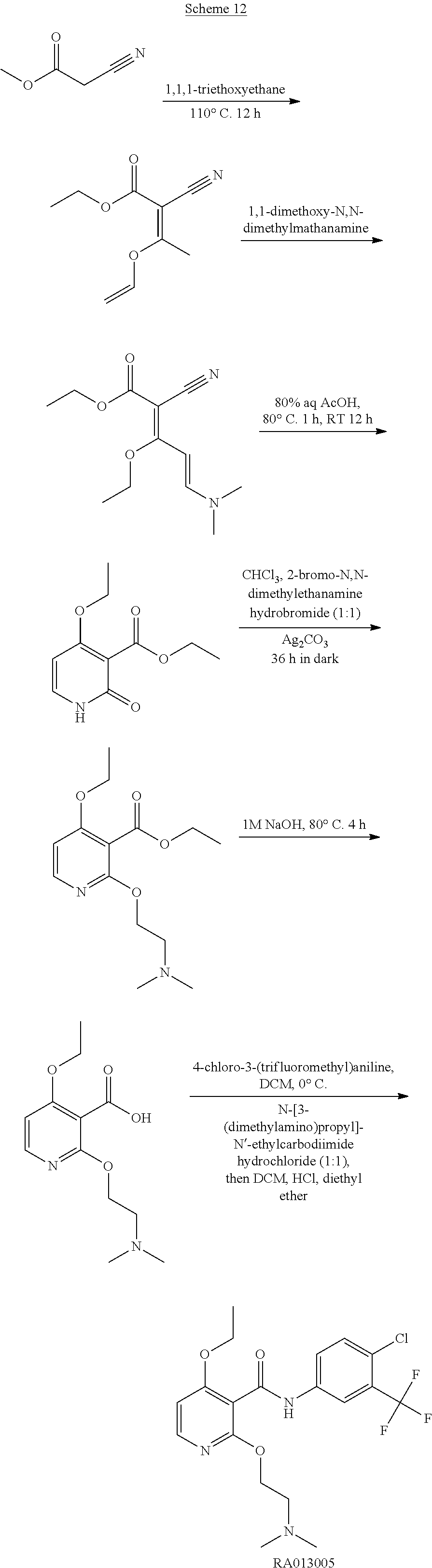

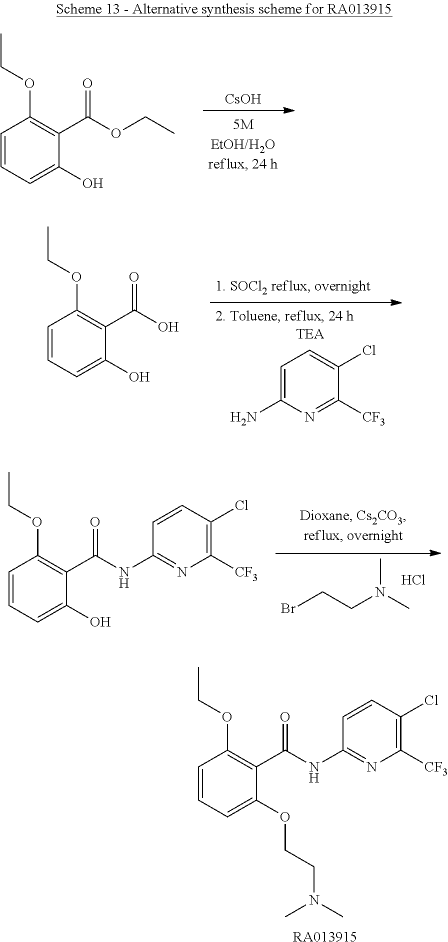

Evaluating the Biological Activity of Compounds of Formula (I)

### Histone Acetyltransferase (HAT) Assay:

The aim of the in vitro acetylation assay is to measure the enzymatic activity of the various compounds towards p300.

First, the drug is prepared:

| | | | | | | | | | | | % of |
|---|---|---|---|---|---|---|---|---|---|---|---|
| Sample No. | 1 | 2 | 3 | 4 | 5 | 6 | 7 | 8 | 9 | 10 | DMSO |
| DMSO stock solutions | 10000 | 1000 | 100 | 50 | 10 | 1 | 0.1 | 0.001 | 0 | Blank | 100% |
| (mM) | | | | | | | | | | | |
| Vol. (μL) of 5xHAT | 18 | 18 | 18 | 18 | 18 | 18 | 18 | 18 | 18 | 18 | |
| assay buffer | | | | | | | | | | | |
| Vol. (μL) of DMSO | 2 | 2 | 2 | 2 | 2 | 2 | 2 | 2 | 2 | / | 2 μl |
| stock solutions in final | | | | | | | | | | | (pure |
| 20 uL of assay buffer | | | | | | | | | | | DMSO) |
| 10x dil. stock in assay | 1000 | 100 | 10 | 5 | 1 | 0.1 | 0.01 | 0.0001 | 0 | Blank | 10% |
| buffer (mM) | | | | | | | | | | | |
| Vol. (μL) of 10x dil. | 2 | 2 | 2 | 2 | 2 | 2 | 2 | 2 | 2 | 2 | |
| stock to final 20 ul | | | | | | | | | | | |
| Final conc. in assay | 100 | 10 | 1 | 0.5 | 0.1 | 0.01 | 0.001 | 0.00001 | 0 | Blank | 1% |
| (mM) | | | | | | | | | | | |
| Final conc. in assay | 100 μM | 10 μM | 1 μM | 500 nM | 100 nM | 10 nM | 1 mM | 0.01 nM | 0 | Blank | |
| (μM) | | | | | | | | | | | |

Second, dilute p300 in AM1 buffer to a concentration of 40 ng/μL (final concentration in the reaction 20 ng/μL). This is accomplished by diluting 1 μL of p300 (at 0.4 μg/μL) into 19 μL of AM1 buffer.

Third, prepare the Master Mix. Prepare the Master Mix in low protein binding tubes (ThermoFisher Cat. No. 90410), 20 μL system.

| | | | 100 | | | | | 0.001 | | |
|---|---|---|---|---|---|---|---|---|---|---|
| stock conc. | 10 mM | 1 mM | μM | 50 μM | 10 μM | 1 μM | 0.1 μM | μM | 0 | Blank |
| DRUG (μL) | from | 10 of | 10 of | 10 of | 10 of | 10 of | 10 of 1 | 10 of | | |
| | powder | 10 | 1 mM | 100 | 50 μM | 10 μM | μM | 0.1 μM | | |
| | | mM | | μM | | | | | | |
| 1% DMSO (μL) | | 90 | 90 | 10 | 40 | 90 | 90 | 990 | 10 | |
| HAT Buffer | | | | | | | | | | |
| final conc. | 100 | 10 | 1 μM | 500 | 100 | 10 nM | 1 nM | 0.01 nM | 0 | Blank |
| | μM | μM | | nM | nM | | | | | |

| Components | 1× | Final conc. in 20 μL |
|---|---|---|
| P300 (20 ng/μL) | 2 μl | |
| 5× HAT buffer | 4 μl | |
| Compound | 2 μl | 0, 0.01, 1, 10, 100, |
| | | 500, 10³, 10^{4,} 10⁵ |

Incubate reactions at 30° C. for 30 minutes.

| | | |
|---|---|---|
| Histone 3.3 | 1 μl | |
| (1 μg/ μl) | | |
| Ac—CoA | 1 μl | 0.025 μM |
| (0.5 μM) | | |
| H2O | 10 μl | |
| TOTAL | 20 μl | |

Incubate reactions at 30° C. for 1 hour.

Fourth, perform the western blot assay.
Add 6.7 μL of Laemmli Sample Buffer 4× (Bio-Rad Laboratories Cat. No. 161-0737) for each reaction and boil samples at 95° C. for 5 min.
Charge 10 μL for each sample in 2 Tris Glycine 4-15% gels (Bio-Rad Laboratories Cat. No. 456-1086). Running Buffer: Tris-Glycine 1× (pour in the cell up to the writing “2 gels”).
Run at 90 V for 1 hour (until the gel front reaches the green line of cell).
Semi-dry transfer with Trans-Blot® Turbo™ Blotting System from Bio-Rad Laboratories (Trans-Blot® Turbo™ RTA midi PVDF transfer kit Cat. No1704275).
   Activate PVDF membrane in methanol for 5 minutes and then wet in Trans-Blot® Turbo™ transfer buffer.
   Wet stacks in transfer buffer.
Transfer at 23 V 1.3 A for 7 minutes.
Blocking buffer: 5% Non-Fat milk in TBST (tween 0.1%) 1 hour at room temperature.
Cut the membranes to get Ac-His 3 at 17 kDa and p300 at 300 kDa.
Incubate with primary antibodies overnight at 4° C.
The day after, make 3 washes in TBST (Tween 0.1%) of 10 minutes.
Incubate with secondary antibody at room temperature for 1 hour.
Make 3 washes in TBST (Tween 0.1%) of 5 minutes.
Incubate membranes with ECL (SuperSignal West Dura Extended Duration Substrate from ThermoFisher) for 5 minutes at room temperature.
Acquire the image with ChemiDoc Odyssey Fc 2 minutes.

To detect total H3 (loading control), strip the membranes with Restore™ Western Blot Stripping Buffer according to the manufacturer protocol.

| | Primary antibody | Secondary antibody |
|---|---|---|
| | dilution | dilution |
| Mouse p300 | 1:1000 | 1:2000 |
| Rabbit H3K27ac | 1:2000 | 1:2000 |
| Rabbit H3K18ac | 1:2000 | 1:2000 |
| Rabbit H3 | 1:5000 | 1:5000 |

### Stability in Human Liver Microsomes

Some compounds were tested for stability in human liver microsomes as shown below in Table 2.

### Experimental Procedure

Mixed-gender human liver microsomes (Lot #1010420) were purchased from XenoTech. The reaction mixture, minus NADPH, was prepared as described below. The test article was added into the reaction mixture at a final concentration of 1 μM. The control compound, testosterone, was run simultaneously with the test article in a separate reaction. An aliquot of the reaction mixture (without cofactor) was equilibrated in a shaking water bath at 37° C. for 3 minutes. The reaction was initiated by the addition of the cofactor, and the mixture was incubated in a shaking water bath at 37° C. Aliquots (100 μL) were withdrawn at 0, 10, 20, 30, and 60 minutes. Test article and testosterone samples were immediately combined with 400 μL of ice-cold 50/50 acetonitrile (ACN)/H2O containing 0.1% formic acid and internal standard to terminate the reaction. The samples were then mixed and centrifuged to precipitate proteins. All samples were assayed by LC-MS/MS using electrospray ionization. Analytical conditions are outlined in Appendix 1. The peak area response ratio (PARR) to internal standard was compared to the PARR at time 0 to determine the percent remaining at each time point. Half-lives and clearance were calculated using GraphPad software, fitting to a single-phase exponential decay equation.

### Reaction Composition

Liver Microsomes 0.5 mg/mL
NADPH (cofactor) 1 mM

### Potassium Phosphate, pH 7.4 100 mM

### Magnesium Chloride 5 mM

### Test Article 1 μM

### APPENDIX 1. ANALYTICAL METHOD

### Liquid Chromatography

### Column: Waters ACQUITY UPLC BEH Phenyl 30×2.1 mm, 1.7 □m

M.P. Buffer: 25 mM ammonium formate buffer, pH 3.5
Aqueous Reservoir (A): 90% water, 10% buffer
Organic Reservoir (B): 90% acetonitrile, 10% buffer
Flow Rate: 0.7 mL/minute

### Gradient Program:

| Time (min) | % A | % B |
|---|---|---|
| 0.00 | 99 | 1 |
| 0.65 | 1 | 99 |
| 0.75 | 1 | 99 |
| 0.80 | 99 | 1 |
| 1.00 | 99 | 1 |

Total Run Time: 1.0 minute
Autosampler: 2 μL injection volume
Wash1: water/methanol/2-propanol:1/1/1; with 0.2% formic acid
Wash2: 0.1% formic acid in water

### Mass Spectrometer

### Instrument: PE SCIEX API 4000

### Interface: Turbo Ionspray

Mode: Multiple reaction monitoring
Method: 1.0 minute duration

TABLE 2

| | | % Remaining of Initial (n = 1) | | | | | | |
|---|---|---|---|---|---|---|---|---|
| | | 0 | 10 | 20 | 30 | 60 | Half-life^{a} | CLᵢₙₜ^{b} (mL/min/ |
| Test Article | Species | min | min | min | min | min | (min) | mg protein) |
| RA010115 | Human | 100 | 47.7 | 21.1 | 11.4 | 2.59 | <10 (9.25) | >0.139 (0.150) |
| RA013915 | Human | 100 | 79.6 | 63.8 | 52.4 | 27.6 | 32.1 | 0.0432 |
| RA013005 | Human | 100 | 40.5 | 14.1 | 4.97 | <1.00 | <10 (7.36) | >0.139 (0.188) |

Intrinsic clearance (CLᵢₙₜ) was calculated based on CLᵢₙₜ=k/P, where k is the elimination constant and P is the protein concentration in the incubation. Of the tested compounds, RA013915 exhibited the best stability with a half-life of 32.1 minutes in human liver microsomes.

### Enzymatic Activity

Various compounds were tested for enzymatic activity with respect to Lys 18 and Lys 27. Results are provided in below in Table 3. EC50 is indicated for Activators and IC50 for inhibitors respectively.

TABLE 3

| | H3-Lys 27 | | H3-Lys 18 | |
|---|---|---|---|---|
| COMPOUND | EC50 | IC50 | EC50 | IC50 |
| RA010115 in DMSO | NO ACTIVITY | NO ACTIVITY | 136.83 nM ± 0.51 | — |
| (n = 6) | | | | |
| RA010143 HCl IN | NO ACTIVITY | NO ACTIVITY | TO BE | — |
| DMSO (n = 3) | | | DETERMINED | |
| RA010146 IN DMSO | NO ACTIVITY | NO ACTIVITY | NO ACTIVITY | NO ACTIVITY |
| (n = 3) | | | | |
| RA010155 IN DMSO | NO ACTIVITY | NO ACTIVITY | 129.06 nM ± 2.01 | — |
| (n = 5) | | | | |
| RA010159 IN DMSO | TO BE | — | NO ACTIVITY | NO ACTIVITY |
| (n = 5) | DETERMINED | | | |
| RA010160 IN DMSO | 532.91 nM ± 1.07 | — | 145.85 nM ± 0.65 | — |
| (n = 5) | | | | |
| RA010162 IN DMSO | NO ACTIVITY | NO ACTIVITY | NO ACTIVITY | NO ACTIVITY |
| (n = 5) | | | | |
| RA010163 IN DMSO | NO ACTIVITY | NO ACTIVITY | NO ACTIVITY | NO ACTIVITY |
| (n = 5) | | | | |
| RA010165 in DMSO | TO BE | — | TO BE | — |
| (n = 3) | DETERMINED | | DETERMINED | |
| RA010168 IN DMSO | NO ACTIVITY | NO ACTIVITY | TO BE | — |
| (n = 3) | | | DETERMINED | |
| RA010171 in DMSO | 6E−141 | — | 141.09 nM ± 1.08 | — |
| (n = 3) | nM ± 327.27 | | | |
| RA010900 HCl in | NO ACTIVITY | NO ACTIVITY | NO ACTIVITY | NO ACTIVITY |
| DMSO (n = 3) | | | | |
| RA010900 HCl in | 43.98 nM ± 0.66 | — | 116.82 nM ± 1.31 | — |
| H2O (n = 3) | | | | |
| RA013005 in DMSO | 593.94 nM ± 1.73 | — | 76.53 nM ± 1.01 | — |
| (n = 5) | | | | |
| RA013011 IN DMSO | — | TO BE | TO BE | — |
| (n = 5) | | DETERMINED | DETERMINED | |
| RA013012 IN DMSO | NO ACTIVITY | NO ACTIVITY | NO ACTIVITY | NO ACTIVITY |
| (n = 5) | | | | |
| RA013886 IN DMSO | NO ACTIVITY | NO ACTIVITY | NO ACTIVITY | NO ACTIVITY |
| (n = 3) | | | | |
| RA013894 IN DMSO | NO ACTIVITY | NO ACTIVITY | NO ACTIVITY | NO ACTIVITY |
| (n = 6) | | | | |
| RA013905 in DMSO | NO ACTIVITY | NO ACTIVITY | NO ACTIVITY | NO ACTIVITY |
| (n = 5) | | | | |
| RA013915 IN DMSO | 57.58 nM ± 1.03 | — | 72.58 nM ± 0.96 | — |
| (n = 5) | | | | |
| RA013917 IN DMSO | 15.91 nM ± 3.16 | — | NO ACTIVITY | |
| (n = 5) | | | | |
| RA013919 IN DMSO | NO ACTIVITY | NO ACTIVITY | TO BE | — |
| (n = 5) | | | DETERMINED | |
| RA013928 IN DMSO | NO ACTIVITY | NO ACTIVITY | NO ACTIVITY | NO ACTIVITY |
| (n = 5) | | | | |
| RA013931 IN DMSO | NO ACTIVITY | NO ACTIVITY | NO ACTIVITY | NO ACTIVITY |
| (n = 5) | | | | |
| RA013938 in DMSO | NO ACTIVITY | NO ACTIVITY | NO ACTIVITY | NO ACTIVITY |
| (n = 5) | | | | |

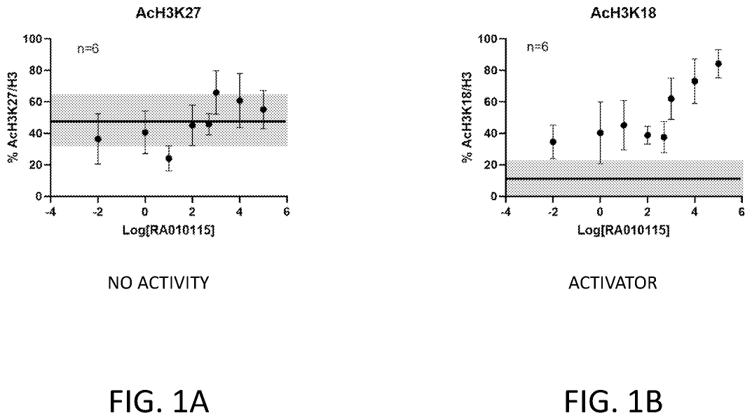

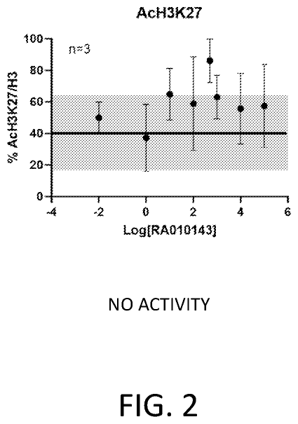

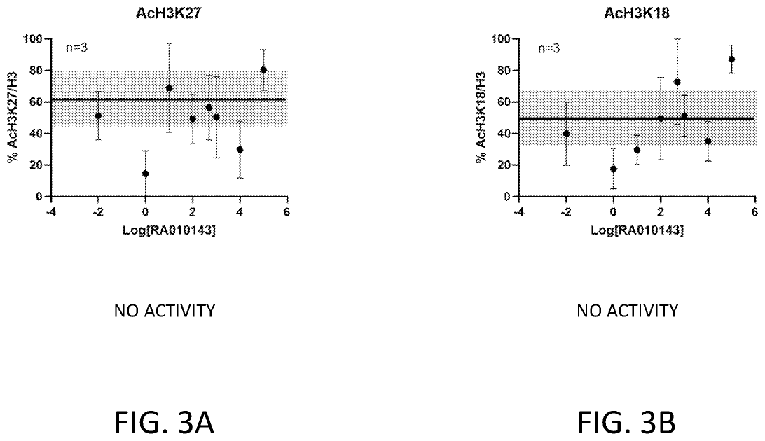

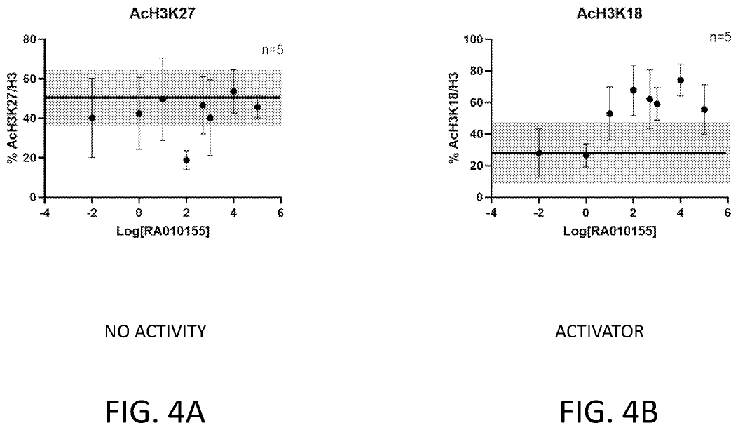

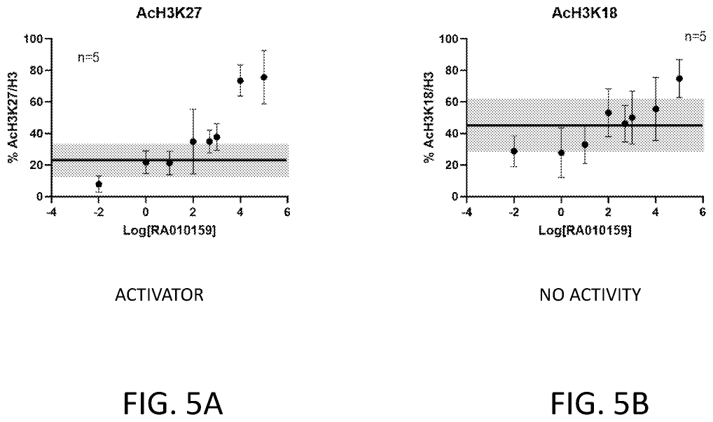

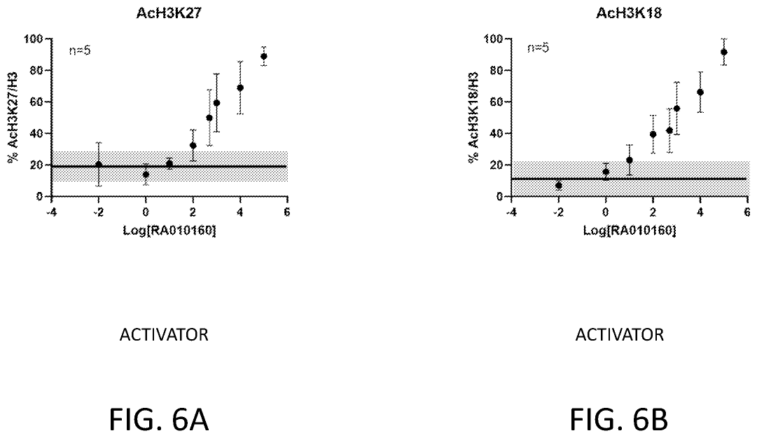

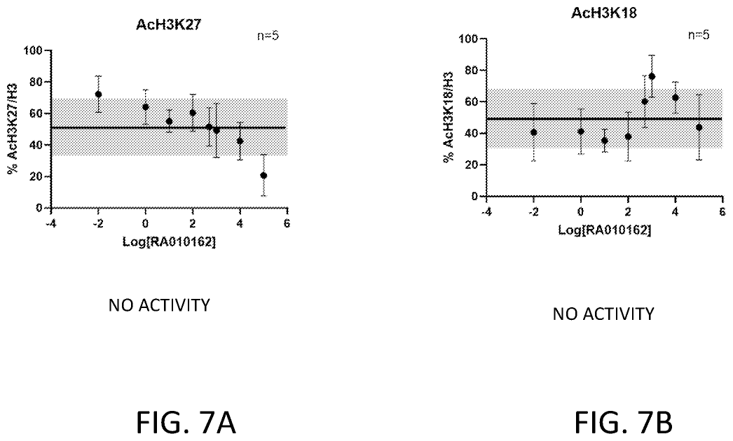

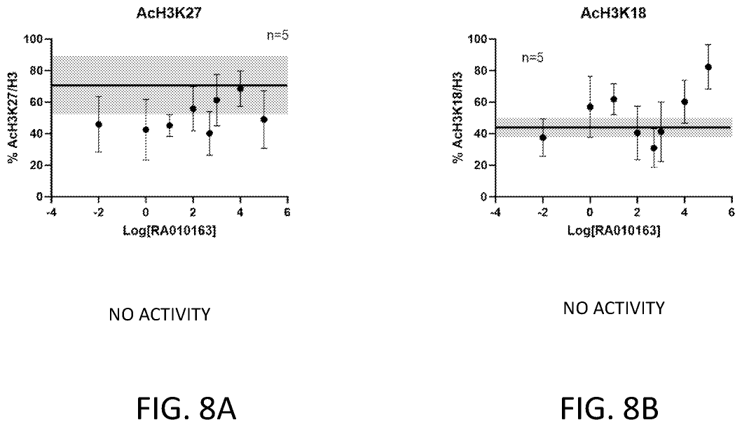

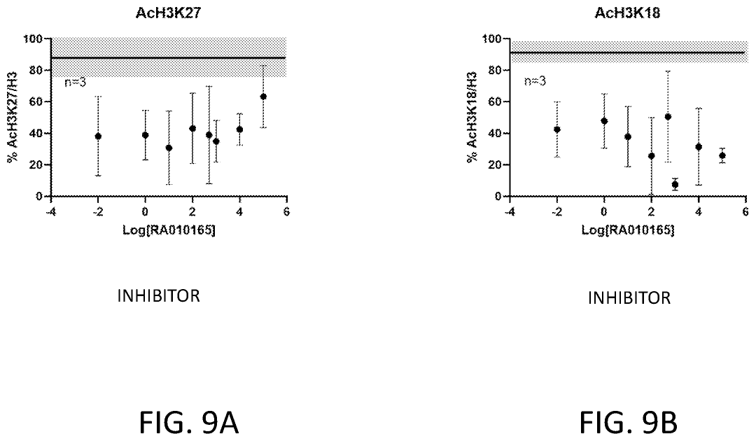

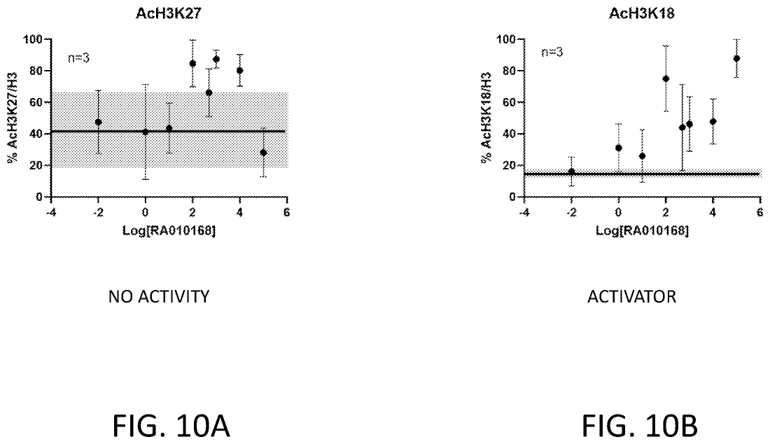

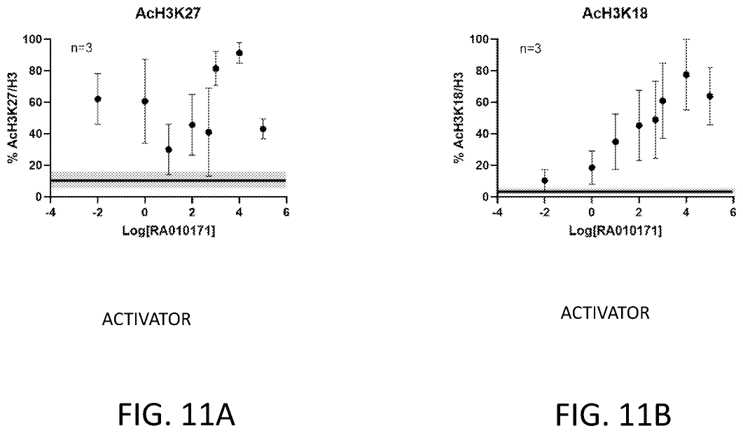

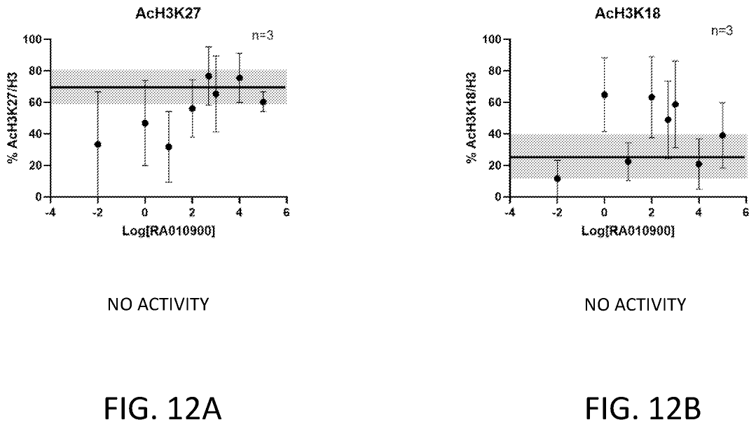

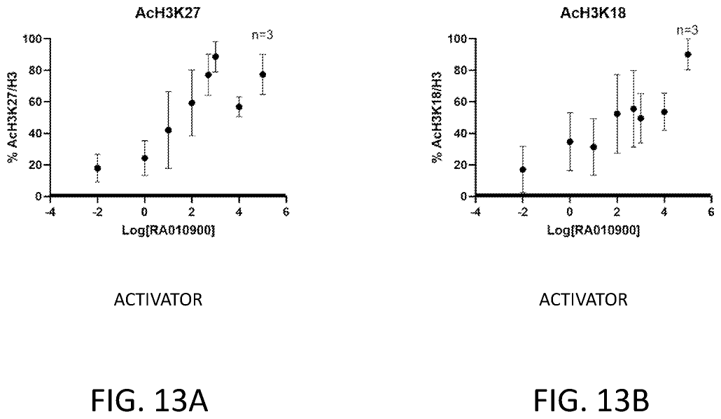

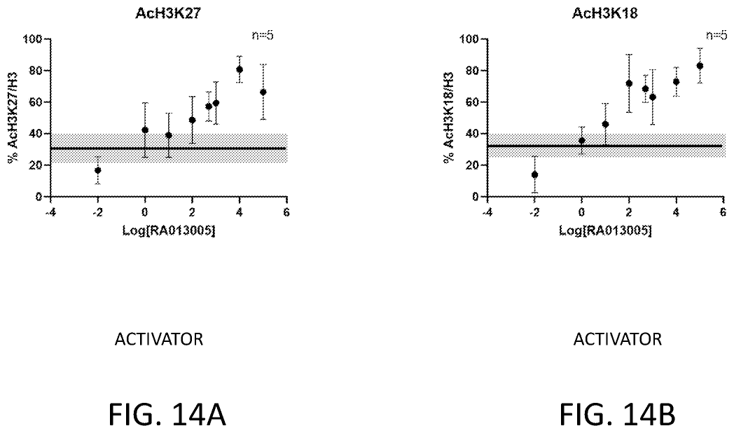

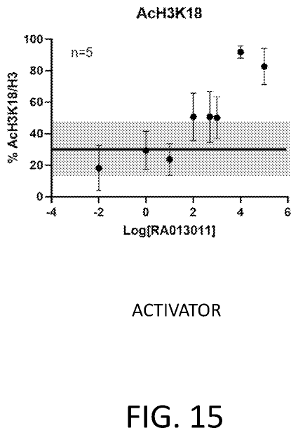

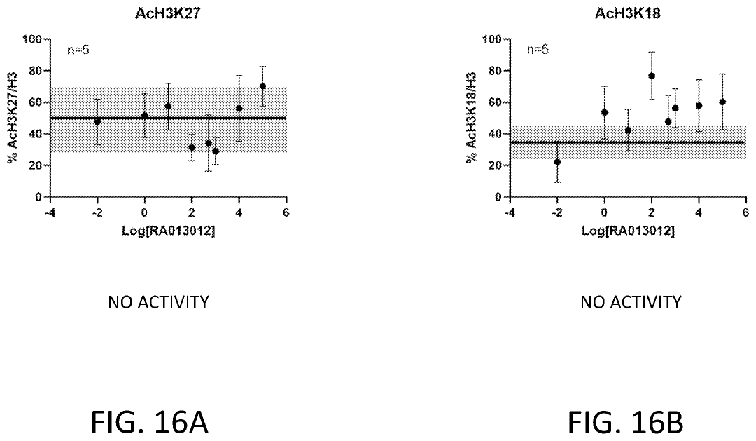

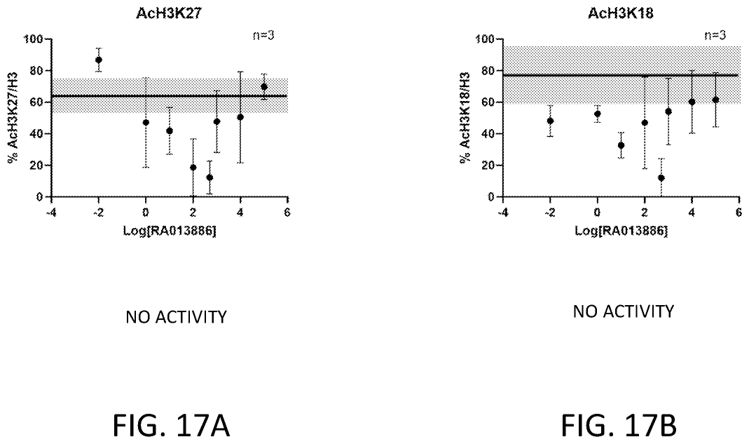

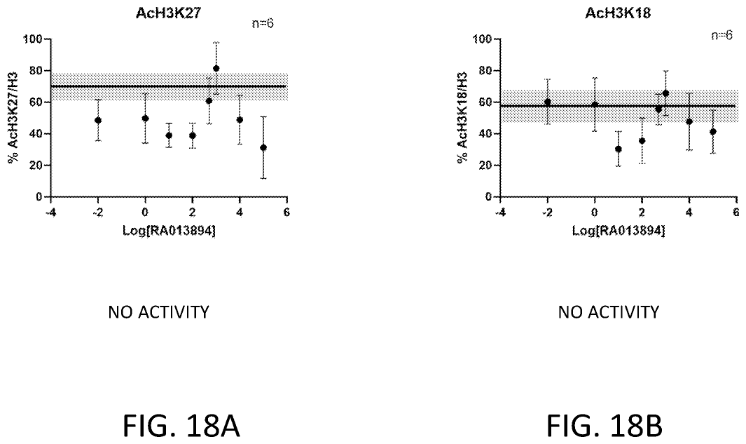

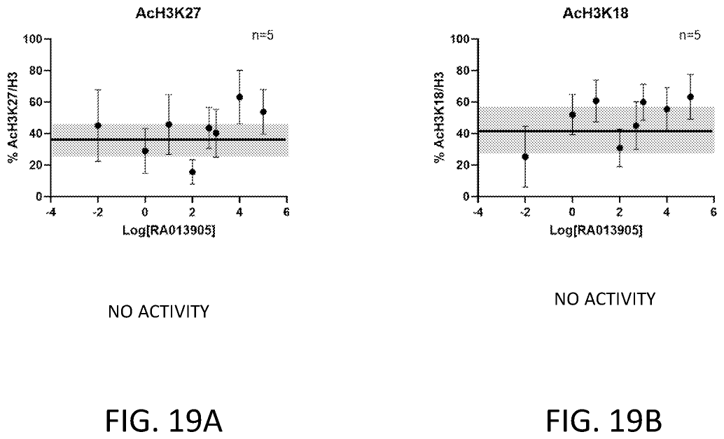

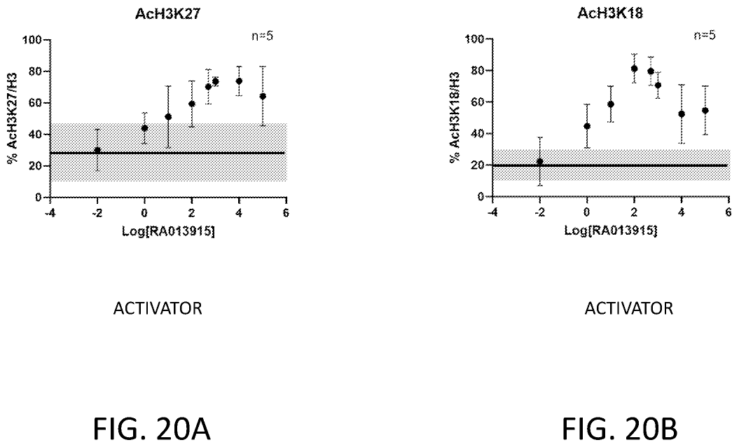

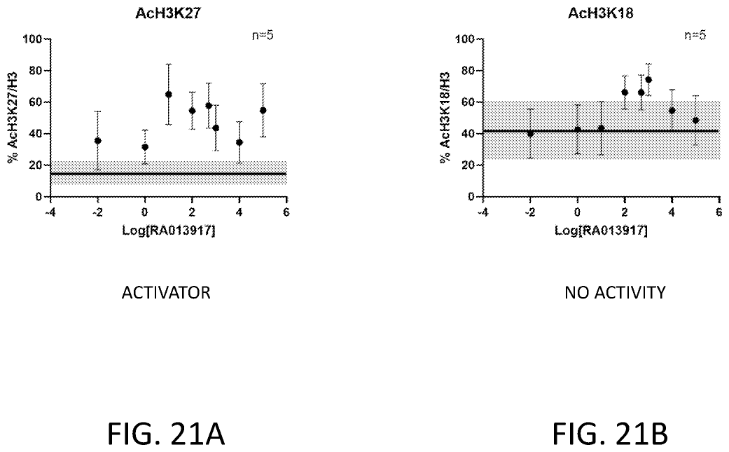

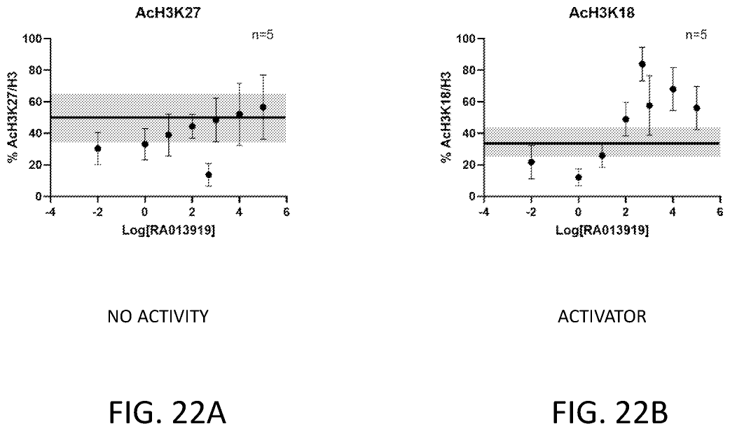

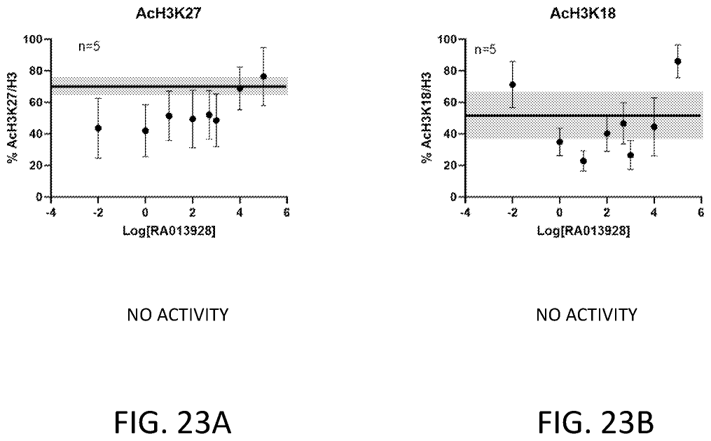

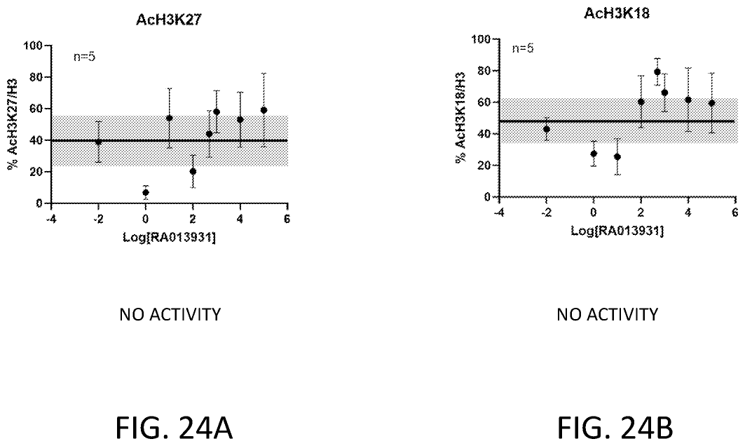

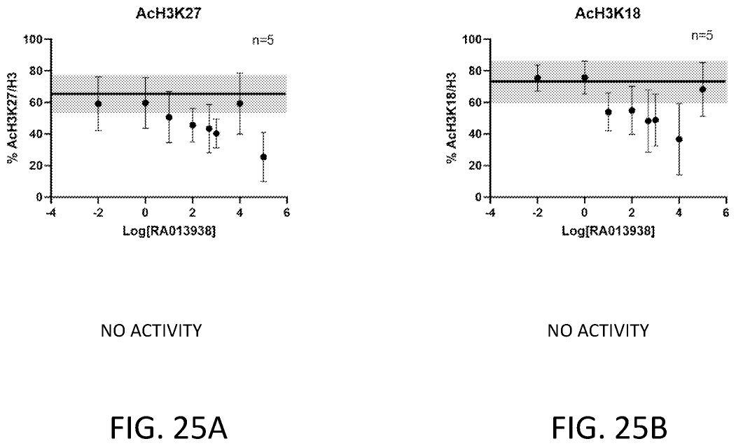

FIGS. 1A to 25B are graphs showing the lysine residue acetylation as a function of concentration for the compounds set forth in Table 3. The shaded area corresponds to the average values of lysine residue acetylation (continuous line) and their standard error range measured in the absence of compound and DMSO. The number of replicates is represented by “n”.

### Tests of Efficacy Against AD Models

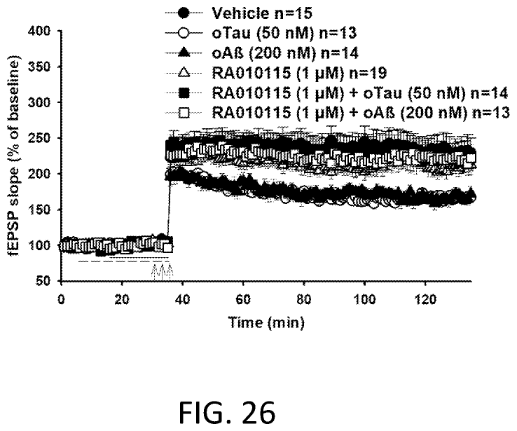

Synaptic dysfunction is a major hallmark of AD (Histol Histopathol, 1995. 10(2): p. 509-19; herein incorporated by reference in its entirety). An aspect of the drug screening protocol can include a measurement of the effect of compounds onto synaptic function. Amyloid-beta (Aβ) is a toxic peptide that is thought to underlie subtle amnesic changes occurring at early stages of Alzheimer's disease. It impairs both memory and its electrophysiological surrogate, long-term potentiation (LTP). LTP can be examined because it is a type of synaptic plasticity thought to underlie learning and memory. RA010115 can rescue the Aβ-induced reduction of LTP, and other compounds can also be screened to identify those that can re-establish normal LTP. The compounds can be applied for 20 min. Controls can be performed on slices in the absence of Aβ, and mice treated with compound. Tau is another peptide that is involved in cell to cell communication, and impairs both memory and LTP in animal models of AD and other tauopathies. FIG. 26 is a graph showing that RA010115 rescues oligomeric Tau (oTau)- and oligomeric Aβ (o Aβ)-induced LTP deficits. LTP was impaired in hippocampal slices from WT mice perfused with oTau (50 nM) and oAβ (200 nM), whereas there was no impairment in slices treated with RA10115 or vehicle. LTP was restored in slices perfused with RA010115 and either oTau or oAβ. The horizontal solid bar represents oAβ and oTau perfusion while the horizontal dashed bar represents RA010115. The three arrows correspond to the theta-burst stimulation. Two-Way ANOVA Vehicle vs. oTau: F(1, 26)=8.119, p=0.00085; Vehicle vs. oAβ: F(1, 27)=8.769, p=0.0063; Vehicle vs. RA010115+oTau: F(1, 27)=0.02696, p=0.8708; Vehicle vs. RA010115+oAβ: F(1, 27)=0.1802, p=0.6747; Vehicle vs. RA010115: F(1, 32)=0.8705, p=0.3578; oTau vs. oAβ: F(1, 25)=0.05339, p=0.8191; oTau vs. RA010115+oTau: F(1, 25)=16.50, p=0.0004; oTau vs. RA010115+oAβ: F(1, 24)=6.665, p=0.0164; oTau vs. RA010115: F(1, 30)=8.312, p=0.0072; oAβ vs. RA010115+oAβ: F(1, 25)=7.433, p=0.0120; oAβ vs. RA010115+oTau: F(1, 26)=20.77, p=0.0001; oAβ vs. RA010115: F(1, 31)=7.539, p=0.0049; RA010115+oAβ vs. RA010115+oTau: F(1, 25)=0.1387, p=0.7127.

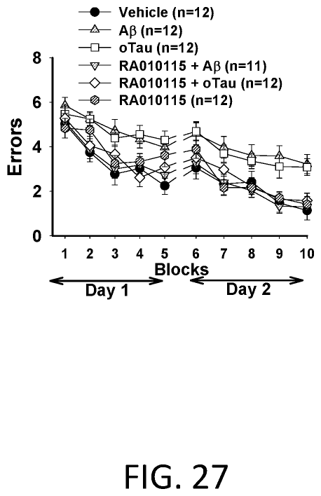

Synaptic plasticity is thought to underlie memory formation. RA010115 was also tested in assays aimed at determining whether the compound can be beneficial to two types of memory, short-term spatial memory that can tested through the 2 day radial arm water maze, and contextual fear memory, a type of associative memory that depends upon hippocampal function and is impaired in AD patients. FIG. 27 is a graph showing that RA010115 rescues oTau- and oAβ-induced defects in the 2-day radial arm water maze test of spatial short-term memory. The performance in the RAWM was impaired in mice administered with oAβ (200 nM) oTau (500 nM). Treatment with the HAT activator RA010115 (5 mg/kg) rescued the deficit. The performance was not impaired when mice were treated with only RA010115 or vehicle. (ANOVA for repeated measures among all groups at day 2: F(5, 65)=7.092, p<0.0001. One-way ANOVA for block 10: F(5, 65)=7.385, p<0.0001; Bonferroni's p<0.0001 for both oAβ vs. vehicle and oTau vs. vehicle; oAβ vs RA010115 plus oAβ p<0.001 and for oTau vs RA010115 plus oTau p<0.05.

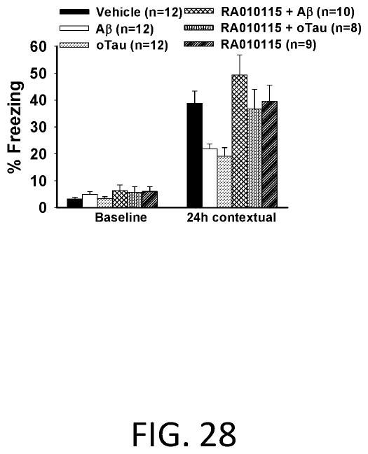

FIG. 28 is a graph showing that RA010115 rescues oTau- and oAβ-induced defects in contextual fear memory. There is statistical significance when comparing all groups during testing for contextual fear memory at 24 hrs after the electric shock (ANOVA among all groups: F(5, 57)=5.558 p=0.0003). Comparisons between groups revealed a statistically significant difference in freezing behavior when comparing mice that received RA010115 plus oTau with oTau-administered animals (t-test: t(18)=2.481, p=0.0232) and mice that received RA010115 plus oAB with oAβ-administered animals (t-test: t(20)=3.907, p=0.0009) Furthermore, oTau-treated mice showed amounts of freezing which were statistically different from vehicle-treated mice (t-test: t(22)=3.518, p=0.0019), and oAβ-treated mice showed amounts of freezing which were statistically different from vehicle-treated mice (t-test: t(22)=3.444, p=0.0023. The performance was not impaired when mice were treated with only RA010115 or vehicle. There were no differences in the baseline freezing between groups (ANOVA: F(5,57)=1.053 p=0.3958).

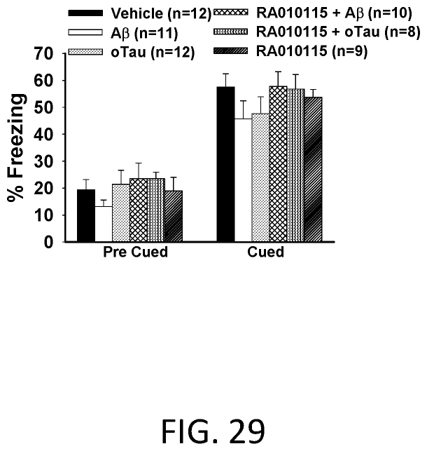

A control is shown on FIG. 29 that displays a graph with the average freezing in cued fear associative memory test in the presence oTau and oAβ with and without RA010115. This test excludes that the effect on contextual fear memory is due to amygdala involvement. No difference was detected between groups in freezing behavior before (pre cue, ANOVA: F(5, 56)=0.7692, p=0.5759) and after (post cue, ANOVA: F(5, 56)=0.938, p=0.4637) the auditory cue in the cued conditioning test.

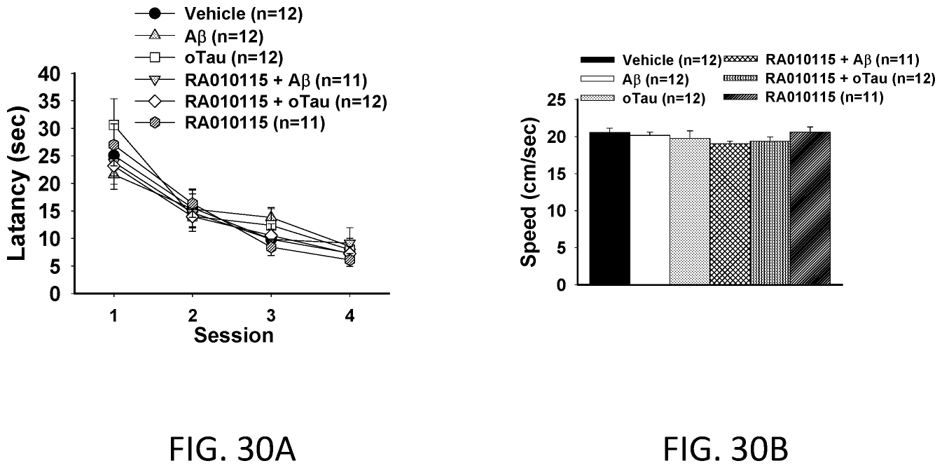

Another control is shown on FIG. 30A and FIG. 30B displaying the average time and speed to reach a platform located above the surface of the water in the presence oTau and oAβ with and without RA010115. No difference was detected between groups for visible platform location (2 way ANOVA: F(5,64)=0.191, p=0.9651) and no difference was detected between groups in swim speed (2 way ANOVA: F(5,64)=0.621, p=0.6845).

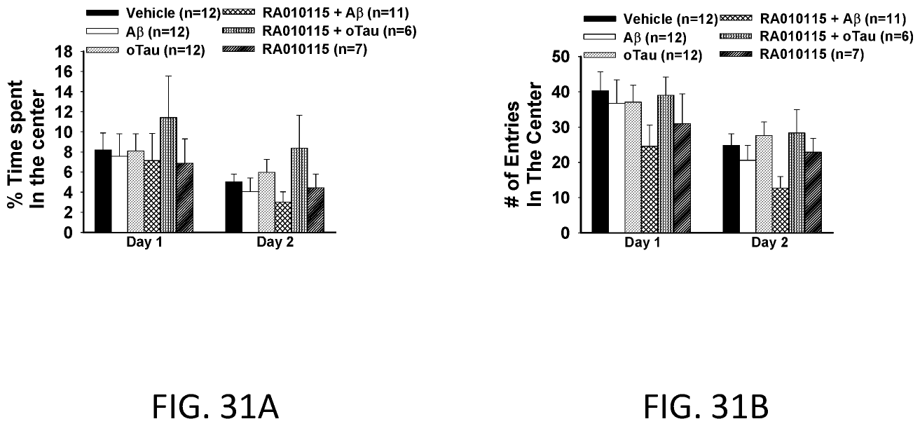

FIG. 31A and FIG. 31B show the performance of mice in the open field test in the presence oTau and oAβ with and without RA010115. Both the time spent in the center of the arena (A) and the number of entries in the center (B) are plotted and indicated that change in anxiety level are not responsible for the beneficial effect of the compound. As shown in FIG. 31A, no differences were observed in the time spent in the center compartment (ANOVA: F(5,54)=0.1385, p=0.244) between groups on the second day in the open field test. Likewise, in FIG. 31B no differences were observed in the number of entries into the center (ANOVA: F(5, 54)=0.205, p=0.0861) between groups on the second day in the open field test.

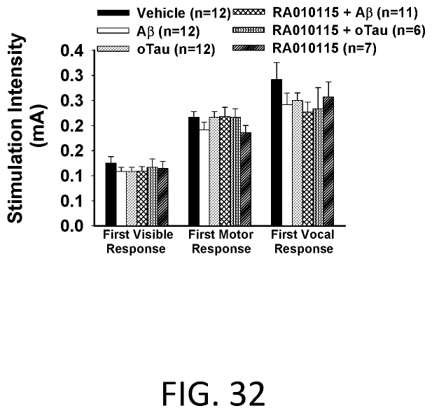

Finally, FIG. 32 shows that the sensory threshold is not affected by the presence oTau and oAβ despite the presence of RA010115, suggesting that the beneficial effect of the compound onto fear memory is not due to changes in the capability of the mouse to perceive the shock, instead of real changes in memory formation. There were no statistically significant differences among groups during the assessment of the sensory threshold (ANOVA among all groups: first visible response F(5, 54)=0.405, p=0.843; motor response F(5, 54)=2.12, p=0.078 and audible response F(5, 54)=0.738, p=0.599).

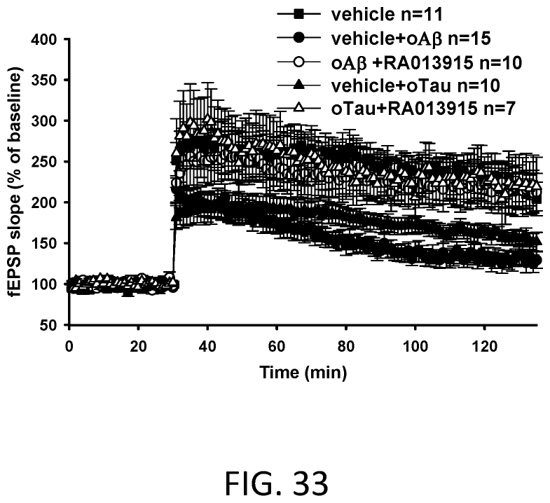

RA013915 is another HAT activator. FIG. 33 displays a graph showing that the compound rescues oligomeric Tau (oTAU)- and Aβ (oAβ)-induced LTP deficits. LTP was impaired in hippocampal slices from WT mice perfused with oTau (50 nM) and oAβ (200 nM), compared to slices treated with vehicle. LTP was restored in slices perfused with RA013915 and either oTau or oAβ. Two-Way ANOVA Vehicle vs. oTau: F(1, 19)=11.14, p=0.0035; Vehicle vs. oAβ: F(1, 24)=22.57, p<0.0001; oTau vs. RA010115+oTau: F(1, 15)=5.552, p=0.0325; oAβ vs. RA010115+oAβ: F(1, 23)=9.977, p=0.0044; The “n” represents the number of slices per condition.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

1. A compound of Formula (I), 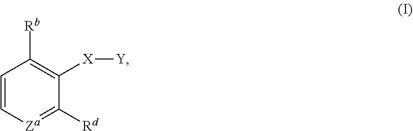
wherein
X is —C(O)N(R^{a1})— or —N(R^{a2})C(O)—;
Y is —C₁₋₆-alkyl 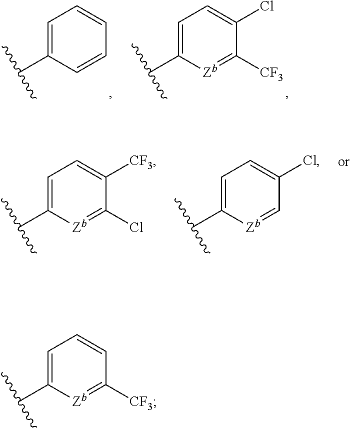
Z^{a} and Z^{b} are each independently CH or N;
R^{a1} and R^{a2} are each independently H, —C₁₋₃ alkyl, —(CH₂)ₘ—R^{c};
R^{b} is H, halogen, —OH, —O—C₁₋₆-alkyl;
R^{c} is —OH, —O-alkyl, —NH(C₁₋₃-alkyl), or —N(C₁₋₃-alkyl)₂;
R^{d} is —OH, —OMe, —OEt, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3,
with the proviso that
when X is —C(O)N(H)—, Y is 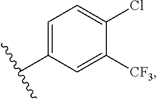 [SMILES: *c1ccc(Cl)c(C(F)(F)F)c1]
Z^{a} is —CH and R^{b} is —OMe or —OEt; R^{d} is not —OH, —O—(CH₂)₂—NMe₂, —O—(CH₂)₂—NEt₂, —O—(CH₂)₃—NMe₂, —O—(CH₂)₃—NEt₂, or —N(H)—(CH₂)₂—NMe₂;
when X is —C(O)N(H)—, Y is 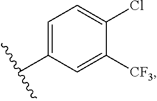 [SMILES: *c1ccc(Cl)c(C(F)(F)F)c1]
Z^{a} is —CH and R^{b} is H; R^{d} is not —OH, —OMe, —OEt, —O—(CH₂)₂—NMe₂ or —N(H)—(CH₂)₂—NMe₂;
when X is —C(O)N(Me)-, Y is 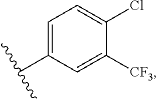 [SMILES: *c1ccc(Cl)c(C(F)(F)F)c1]
Z^{a} is —CH and R^{b} is —OEt; R^{d} is not —O—(CH₂)₂—NMe₂; and
when X is —C(O)N(Me)-, Y is 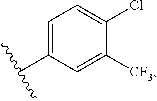 [SMILES: *c1ccc(Cl)c(C(F)(F)F)c1]
Z^{a} is —CH and R^{b} is —OH; R^{d} is not —OH.

2. The compound of claim 1, wherein
X is —C(O)N(R^{a1})— or —N(R^{a2})C(O)—;
Y is —C₁₋₆-alkyl, 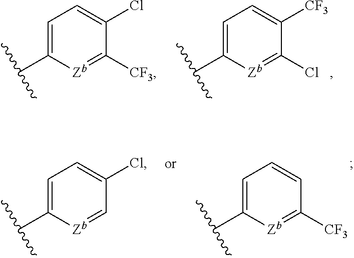
Z^{a} and Z^{b} are each independently CH or N;
R^{a1} and R^{a2} are each independently H, —C₁₋₃-alkyl, —(CH₂)ₘ—R^{c};
R^{b} is H, halogen, —OH, —OMe, —OEt, —OPr, —OiPr, or OBu;
R^{c} is —OH, —O-alkyl, or —N(C₁₋₃ alkyl)₂;
R^{d} is —OH, —OMe, —OEt, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3.

3. The compound of claim 1, wherein
X is —C(O)N(R^{a1})— or —N(R^{a2})C(O)—;
Y is Me 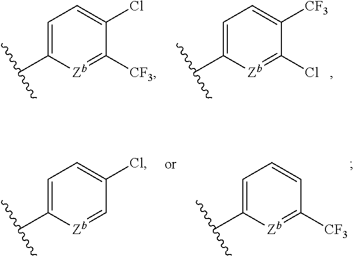
Z^{a} and Z^{b} are each independently CH or N;
R^{a1} and R^{a2} are each independently H, —C₁₋₃-alkyl, —(CH₂)ₘ—R^{c};
R^{b} is H, halogen, —OH, —OMe, —OEt, —OPr, —OiPr, or OBu;
R^{c} is —OH, —O-alkyl, or —N(C₁₋₃ alkyl)₂;
R^{d} is —OH, —OMe, —OEt, —O—(CH₂)ₙ—R^{e1}, —N(H)—(CH₂)ₙ—R^{e2}; or —N(Me)-(CH₂)ₙ—R^{e2};
R^{e1} and R^{e2} are each independently —OH, —OMe, —NH₂, —NHMe, —NMe₂, —NHEt, or —NEt₂;
m is 1, 2, or 3; and
n is 2 or 3.

4. The compound of claim 1, wherein Z^{a} and Z^{b} are CH.

5. The compound of claim 1, wherein X is —C(O)N(R^{a1})— and R^{a1} is H or Me.

6. The compound of claim 1, wherein R^{b} is —OMe, —OEt, —OPr, or —OiPr.

7. The compound of claim 1, wherein Rei is —NMe₂ or —NEt₂.

8. The compound of claim 1, wherein n is 2.

9. The compound of claim 1, having the structure:

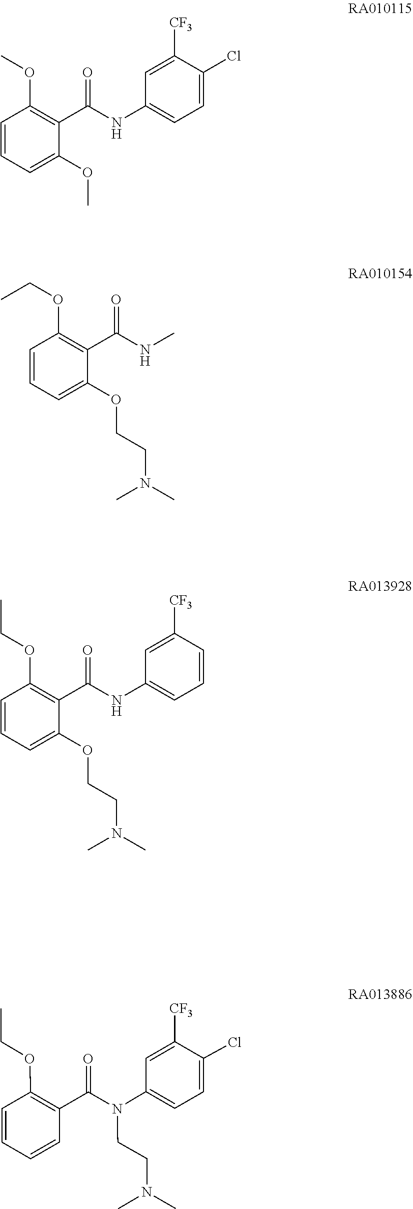 [SMILES: COc1cccc(OC)c1C(=O)Nc1ccc(Cl)c(C(F)(F)F)c1 ; CCOc1cccc(OCCN(C)C)c1C(=O)NC ; CCOc1cccc(OCCN(C)C)c1C(=O)Nc1cccc(C(F)(F)F)c1 ; CCOc1ccccc1C(=O)N(CCN(C)C)c1ccc(Cl)c(C(F)(F)F)c1]

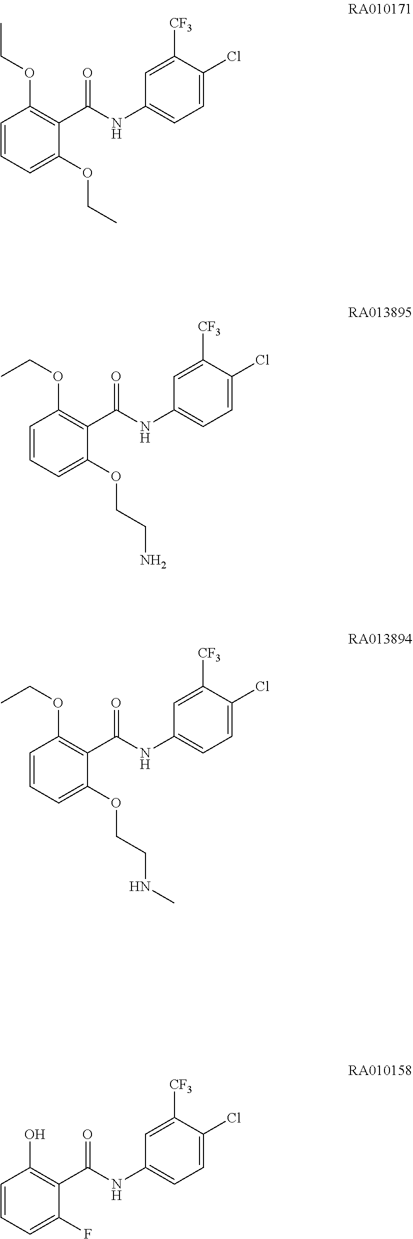 [SMILES: CCOc1cccc(OCC)c1C(=O)Nc1ccc(Cl)c(C(F)(F)F)c1 ; CCOc1cccc(OCCN)c1C(=O)Nc1ccc(Cl)c(C(F)(F)F)c1 ; CCOc1cccc(OCCNC)c1C(=O)Nc1ccc(Cl)c(C(F)(F)F)c1 ; O=C(Nc1ccc(Cl)c(C(F)(F)F)c1)c1c(O)cccc1F]

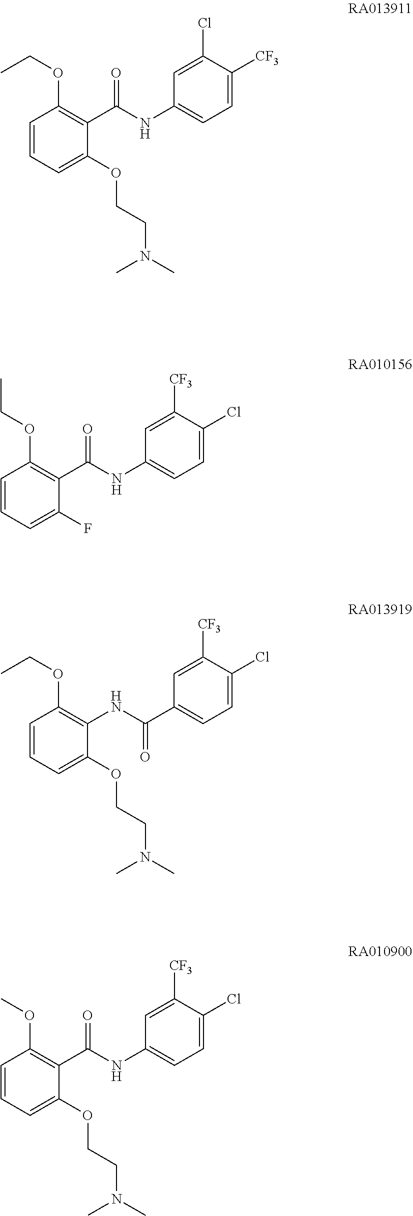 [SMILES: CCOc1cccc(OCCN(C)C)c1C(=O)Nc1ccc(C(F)(F)F)c(Cl)c1 ; CCOc1cccc(F)c1C(=O)Nc1ccc(Cl)c(C(F)(F)F)c1 ; CCOc1cccc(OCCN(C)C)c1NC(=O)c1ccc(Cl)c(C(F)(F)F)c1 ; COc1cccc(OCCN(C)C)c1C(=O)Nc1ccc(Cl)c(C(F)(F)F)c1]

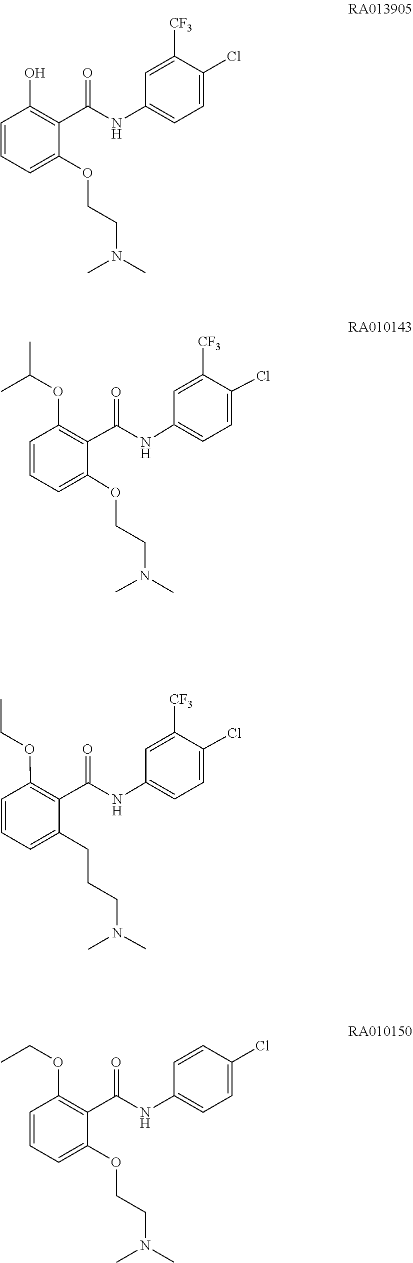 [SMILES: CN(C)CCOc1cccc(O)c1C(=O)Nc1ccc(Cl)c(C(F)(F)F)c1 ; CC(C)Oc1cccc(OCCN(C)C)c1C(=O)Nc1ccc(Cl)c(C(F)(F)F)c1 ; CCOc1cccc(CCCN(C)C)c1C(=O)Nc1ccc(Cl)c(C(F)(F)F)c1 ; CCOc1cccc(OCCN(C)C)c1C(=O)Nc1ccc(Cl)cc1]

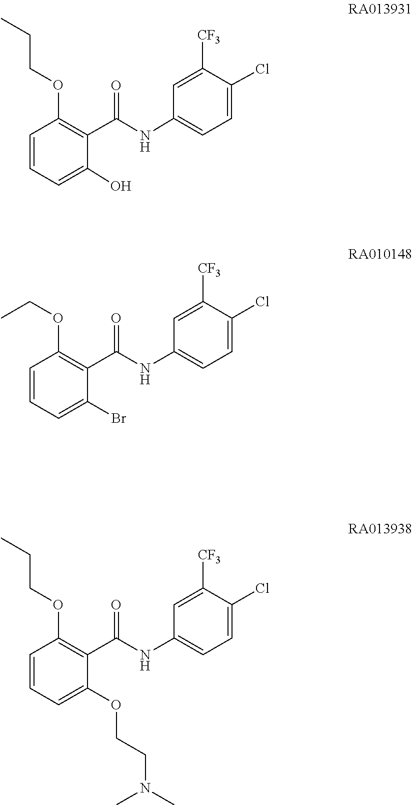 [SMILES: CCCOc1cccc(O)c1C(=O)Nc1ccc(Cl)c(C(F)(F)F)c1 ; CCOc1cccc(Br)c1C(=O)Nc1ccc(Cl)c(C(F)(F)F)c1 ; CCCOc1cccc(OCCN(C)C)c1C(=O)Nc1ccc(Cl)c(C(F)(F)F)c1]

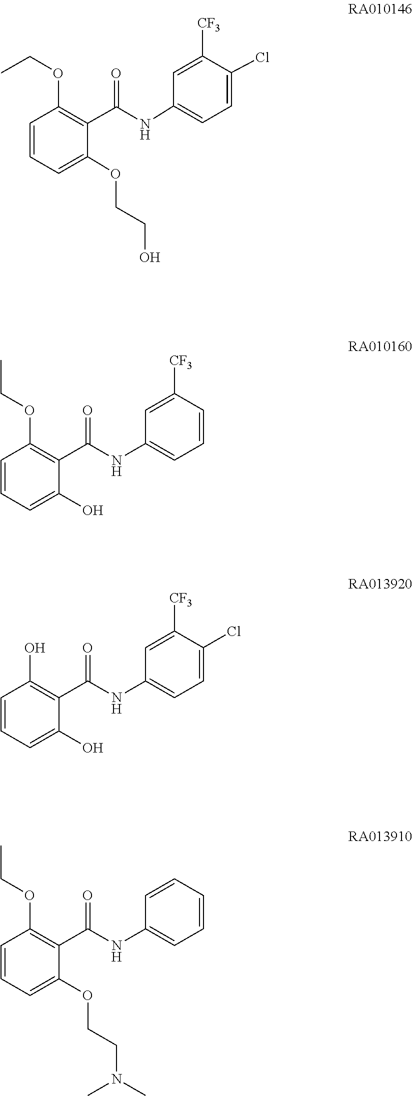 [SMILES: CCOc1cccc(OCCO)c1C(=O)Nc1ccc(Cl)c(C(F)(F)F)c1 ; CCOc1cccc(O)c1C(=O)Nc1cccc(C(F)(F)F)c1 ; O=C(Nc1ccc(Cl)c(C(F)(F)F)c1)c1c(O)cccc1O ; CCOc1cccc(OCCN(C)C)c1C(=O)Nc1ccccc1]

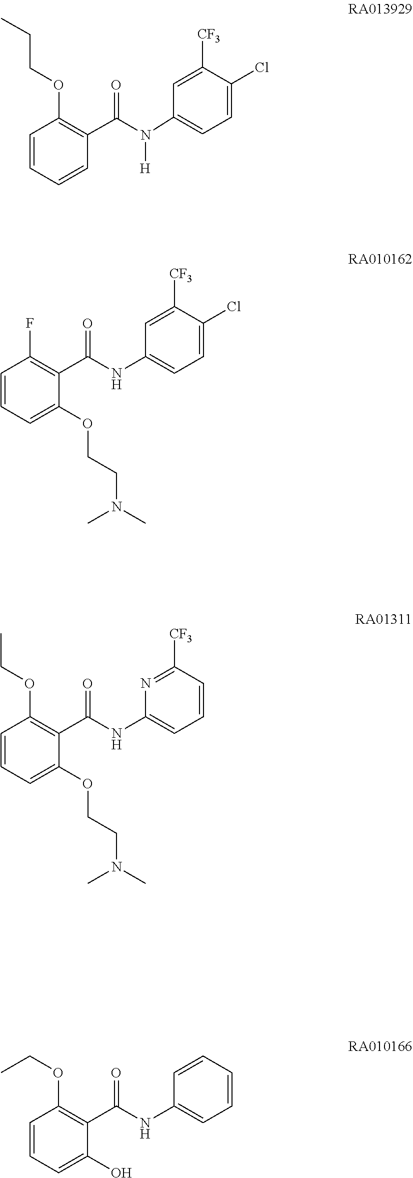 [SMILES: CCCOc1ccccc1C(=O)Nc1ccc(Cl)c(C(F)(F)F)c1 ; CN(C)CCOc1cccc(F)c1C(=O)Nc1ccc(Cl)c(C(F)(F)F)c1 ; CCOc1cccc(OCCN(C)C)c1C(=O)Nc1cccc(C(F)(F)F)n1 ; CCOc1cccc(O)c1C(=O)Nc1ccccc1]

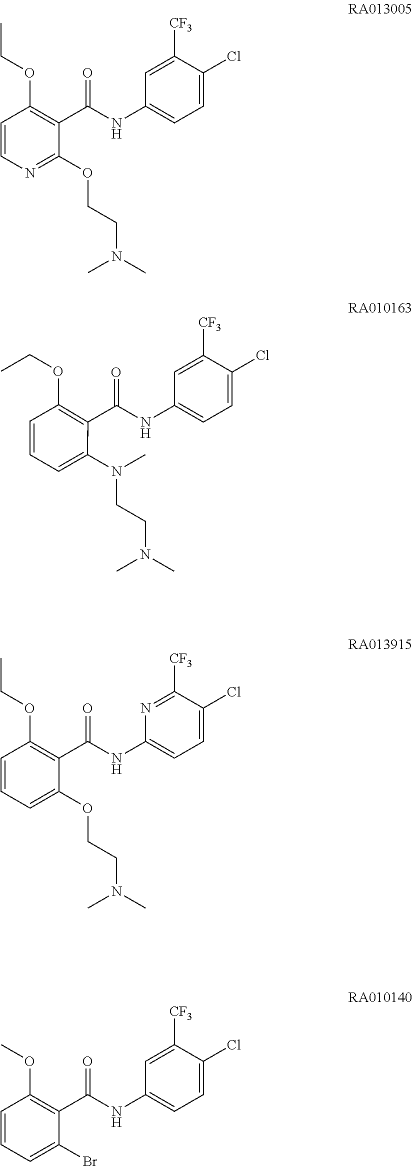 [SMILES: CCOc1ccnc(OCCN(C)C)c1C(=O)Nc1ccc(Cl)c(C(F)(F)F)c1 ; CCOc1cccc(N(C)CCN(C)C)c1C(=O)Nc1ccc(Cl)c(C(F)(F)F)c1 ; CCOc1cccc(OCCN(C)C)c1C(=O)Nc1ccc(Cl)c(C(F)(F)F)n1 ; COc1cccc(Br)c1C(=O)Nc1ccc(Cl)c(C(F)(F)F)c1]

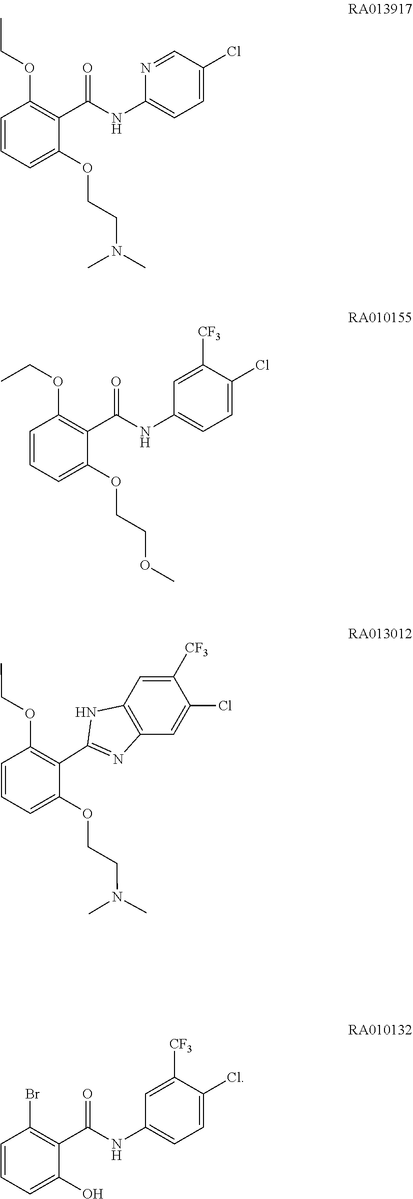 [SMILES: CCOc1cccc(OCCN(C)C)c1C(=O)Nc1ccc(Cl)cn1 ; CCOc1cccc(OCCOC)c1C(=O)Nc1ccc(Cl)c(C(F)(F)F)c1 ; CCOc1cccc(OCCN(C)C)c1-c1nc2cc(Cl)c(C(F)(F)F)cc2[nH]1 ; O=C(Nc1ccc(Cl)c(C(F)(F)F)c1)c1c(O)cccc1Br]

10. A pharmaceutical composition comprising a compound of claim 1 and a pharmaceutically acceptable excipient.

11. The pharmaceutical composition of claim 10, wherein the compound is a HAT activator.

12. The pharmaceutical composition of claim 10, wherein the compound is a HAT inhibitor.

13. A method of increasing histone acetylation in a subject, the method comprising administering to the subject a therapeutically effective amount of a compound of claim 1.

14. The method of claim 13, wherein histone acetylation occurs at K18 and/or K27 of histone 3.

15. A method of treating a neurodegenerative disease is a subject in need thereof, comprising administering to the subject a therapeutically effective amount of a compound of claim 1.

16. A method of improving long term memory formation in a subject afflicted with a neurodegenerative disease or condition, the method comprising administering to the subject a therapeutically effective amount of a compound of claim 1.

17. A method of enhancing memory retention in a subject afflicted with a neurodegenerative disease comprising administering to the subject a therapeutically effective amount of a compound of claim 1.

18. A method of enhancing learning or memory in a subject afflicted with a neurodegenerative disease comprising administering to the subject a therapeutically effective amount of a compound of claim 1.

19. The method of claim 13, wherein the subject is not afflicted with a neurodegenerative disease.

20. The method of claim 15, wherein the neurodegenerative disease is Adrenoleukodystrophy (ALD), Alcoholism, Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, argyrophilic grain disease (AGD), and globular glial tauopathy (GGT), the neurofibrillary tangle-predominant senile dementia (now included also in the category of primary age-related tauopathy, PART), Behavioral variant frontotemporal dementia; Semantic variant primary progressive aphasia, non-fluent/agrammatic variant primary progressive aphasia, logopenic variant primary progressive aphasia, Creutzfeldt-Jakob disease, Familial fatal insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Rett's syndrome, Tau-positive Pronto Temporal dementia, Tau-negative Frontotemporal dementia, Refsum's disease, Sandhoff disease, Schilder's disease, Subacute combined degeneration of spinal cord secondary to Pernicious Anaemia, Spielmeyer-Vogt-Sjogren-Batten disease, Batten disease, Spinocerebellar ataxia, Spinal muscular atrophy, Steele-Richardson-Olszewski disease, Tabes dorsalis, or Toxic encephalopathy.

21. The method of claim 20, wherein the neurodegenerative disease is Alzheimer's Disease, Parkinson's Disease, or Huntington's Disease.

22. A method of treating cancer in a subject in need thereof, the method comprising administering to the subject a therapeutically effective amount of a compound of claim 1.

23. The method of claim 22, wherein the cancer is B cell lymphoma, colon cancer, lung cancer, renal cancer, bladder cancer, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkin's lymphoma, Hodgkin's lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primaiy or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, multiple myeloma, follicular lymphoma or medullary carcinoma

24. The method of claim 22, wherein the cancer is Hodgkin's lymphoma, non-Hodgkin's lymphoma, B cell lymphoma, T cell lymphoma, follicular lymphoma, T cell leukemia, acute myeloid leukemia, acute lymphocytic leukemia, or myeloma.

25. The method of claim 15, wherein the subject has at least one mutant HAT enzyme gene.
